(19) United States
(12) Patent Application Publication
(10) Pub. No.: US 2025/0180581 A1
(43) Pub. Date: Jun. 5, 2025

(54) COMPOSITIONS AND METHODS FOR CHARACTERIZING AND TREATING PSYCHIATRIC ILLNESS

(71) Applicant: Trustees of Tufts College, Medford, MA (US)

(72) Inventor: Jamie Maguire, Medford, MA (US)

(73) Assignee: Trustees of Tufts College, Medford, MA (US)

(21) Appl. No.: 18/965,527

(22) Filed: Dec. 2, 2024

Related U.S. Application Data
(60) Provisional application No. 63/605,546, filed on Dec. 3, 2023.

(51) Int. Cl. G01N 33/68; C12Q 1/6883

(52) U.S. Cl. CPC G01N 33/6893; C12Q 1/6883; C12Q 2600/158; G01N 2800/304; G01N 2800/52

(57) ABSTRACT

Various embodiments of the disclosure are directed to methods for identifying a subject responsive to treatment of a psychiatric illness, diagnosing and treating a psychiatric illness in a subject, determining the efficacy of a medicament for treating a psychiatric illness in a subject, a panel of biomarkers for use in the methods thereof, and compositions for treating a psychiatric illness in a subject. The biomarkers and methods using such biomarkers described here can identify subsets or populations of patients having impaired endogenous neurosteroidogenesis, where the biomarkers serve as predictive biomarkers for identifying patients having a psychiatric illness who are responsive to neuroactive steroid (NAS)-based treatments.

### CROSS REFERENCE TO RELATED APPLICATION

The present application claims priority to and the benefit of U.S. App. No. 63/605,546, filed Dec. 3, 2023, the contents of which is hereby incorporated by reference in its entirety.

### SEQUENCE LISTING

The present application contains a Sequence Listing which has been submitted electronically in XML format and is hereby incorporated by reference in its entirety. Said XML copy, created on Jan. 20, 2025, is named 167774-013601_US_SL.xml and is 24,845 bytes in size.

### BACKGROUND

Psychiatric illnesses affect millions of people globally and as such, there is a need for novel, rapid, and more effective treatments. Treatments currently used for the treatment of psychiatric illnesses include, for example, psychotropic medications, neurostimulation methods, and evidence-based forms of psychotherapy. However, these are only somewhat beneficial to patients. These treatments provide delayed benefits if any, and responses are often partial and limited by side effects, poor patient compliance, and relapse. Additionally, due to the heterogeneity in the psychiatric patient population, identifying beneficial treatment for individual patients is challenging. There is a long felt and unmet need for biomarkers that can characterize psychiatric illnesses in individuals, determine the risk for psychiatric illnesses in an individual, and identify and/or stratify individuals suffering from a psychiatric illness within a heterogeneous population to identify effective therapies.

### SUMMARY

The disclosure provides compositions and methods for characterizing psychiatric illness (e.g., depression) associated with neurosteroidogenic pathways, and therapy selection.

In some aspects, a panel of polypeptide or polynucleotide biomarkers are provided. The biomarkers useful in such panels are such as those selected from the group consisting of: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKR1C4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

Other aspects provide a method of treating a subject selected as having a reduced capacity for neurosteroidogenesis, where the method comprises administering to the selected subject either exogenous neurosteroids or an agent that increases endogenous neurosteroidogenesis, where the subject is selected by detecting a reduction in a biomarker in a sample of the subject relative to a reference. In further aspects, disclosed here are methods of characterizing a subject having or having a propensity to develop a psychiatric illness, the method comprising measuring levels of a biomarker in a sample from the subject compared to a reference, thereby characterizing the subject as having or having a propensity to develop a psychiatric illness. Another aspect provides a method of determining the efficacy of a medicament or monitoring the efficacy of a medicament for treating a psychiatric illness in a subject, the method comprising: a) measuring levels of a biomarker in a sample from the subject prior to administration of the medicament; b) measuring levels of the biomarkers in a second sample from the subject following treatment with the medicament, where an increase in the level of the biomarker in the first sample relative to the second sample indicates that the medicament is efficacious for treating the psychiatric illness. In some aspects, the measuring comprises a) isolating total protein from the sample from the subject and from a reference sample; b) detecting antibody binding to a biomarker present in the sample of the subject relative to antibody binding to the reference sample; and c) quantifying protein expression. Other aspects provide methods where the measuring comprises: isolating RNA from the sample from the subject and from the sample from the reference; adding biomarker-specific primers and test control-specific primers (e.g., β-actin; GAPDH); and quantifying levels of expression. Further aspects provide a method of treating a subject having a psychiatric illness characterized by reduced levels of one or more biomarkers of endogenous neurosteroidogenesis, comprising: (a) detecting expression of the one or more biomarkers in a sample obtained from the subject by contacting the sample with an antibody against the one or more biomarkers and determining if the antibody bound to the one or more biomarkers; (b) comparing expression of the one or more biomarkers in the sample to a reference from a healthy control subject; and (c) administering a neuroactive steroid-based agent to the subject if the sample has reduced levels of one or more biomarkers of endogenous neurosteroidogenesis as compared to the reference.

Additional aspects of the disclosure provide a kit is provided that comprises a capture molecule that specifically binds a biomarker; and/or a primer set and a detectably labeled probe that specifically amplifies and detects at least one or more biomarkers.

In any of the aspects described and provided here, the biomarker useful in any such panels, kits, and methods include those selected from the group consisting of steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKRIC2); aldo-keto reductase family 1, member C3 (AKRIC3); aldo-keto reductase family 1, member C4 (AKRIC4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

Other aspects provide administering to a subject having a reduced capacity for neurosteroidogenesis, a treatment, a medicament, or a therapeutic agent in an effective amount to increase the levels of endogenous neuroactive steroids, metabolites, intermediaries, and neurosteroidogenic enzymes. In further aspects, the medicament or therapeutic agent is an activator or inducer of or increases endogenous neurosteroidogenesis.

Compositions and articles defined by the disclosure were isolated or otherwise manufactured in connection with the examples provided below. Other features and advantages of the disclosure will be apparent from the detailed description, and from the claims.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a polypeptide, nucleic acid molecule, or small compound. In embodiments, the agent increases neurosteroidogenesis in the subject.

By “ameliorate” is meant decrease, suppress, attenuate, diminish, arrest, or stabilize the development or progression of a disease.

By “alteration” or “modification” is meant a change (increase or decrease) in the expression levels, structure, or activity of a gene or polypeptide as detected by standard art known methods such as those described herein. As used herein, an alteration can include a 10% change in expression levels, a 25% change, a 40% change, or a 50% or greater change in expression levels.

By “capture reagent” is meant a reagent that specifically binds a nucleic acid molecule or polypeptide to select or isolate the nucleic acid molecule or polypeptide.

By “biomarker” or “marker” is meant any protein or polynucleotide having an alteration in expression level or activity that is associated with a disease or disorder. For example, a biomarker as used here, refers to a biological compound, such as a protein or polynucleotide, that is differentially expressed in a sample obtained from a subject having or at risk of having a psychiatric illness as compared to the level present in a reference. In one embodiment, the reference is a comparable sample obtained from a control subject (i.e., a person who is normal or healthy, or a person with a negative diagnosis) or a reference control containing a biomarker at a level found in such a control subject. The biomarker can be a protein, a nucleic acid or fragment thereof, a polynucleotide, or an oligonucleotide, where the biomarker is detectable and/or quantifiable. In embodiments, biomarkers described herein are useful for characterizing a patient sample and in diagnostics. The accuracy of a diagnostic test can be characterized using any method well known in the art, including, but not limited to, a Receiver Operating Characteristic curve (“ROC curve”). An ROC curve shows the relationship between sensitivity and specificity. Sensitivity is the percentage of true positives that are predicted by a test to be positive, while specificity is the percentage of true negatives that are predicted by a test to be negative. An ROC is a plot of the true positive rate against the false positive rate for the different possible cutpoints of a diagnostic test. Thus, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test. In embodiments, biomarkers and diagnostic methods of the present disclosure have an AUC greater than 0.50, greater than 0.60, greater than 0.70, greater than 0.80, or greater than 0.9. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

“Detect” refers to identifying the presence, absence or amount of the analyte to be detected.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. Methods for labeling and guidance in the choice of labels appropriate for various purposes are discussed, e.g., in Green, M. R., Hughes, H., Sambrook, J., & MacCallum, P. (2012). Molecular cloning: a laboratory manual. In Molecular cloning: a laboratory manual and Ausubel et al., Current Protocols in Molecular Biology. (1998). United States: John Wiley & Sons. The attachment of a compound (e.g., an antibody) to a label may be through covalent bonds, adsorption processes, hydrophobic and/or electrostatic bonds, as in chelates and the like, or combinations of these bonds and interactions and/or may involve a linking group.

By “disease” is meant any condition or disorder that damages or interferes with the normal function of a cell, tissue, or organ.

“Diagnosis” as used here generally refers to a determination as to whether a subject is likely affected by a given disease, such as a psychiatric illness. A skilled artisan can make a diagnosis on the basis of one or more diagnostic indicators, such as a biomarker, the presence, absence, or amount of which is indicative of the presence or absence of the disease.

By “marker profile” is meant a characterization of the expression or expression level of two or more polypeptides or polynucleotides.

By “prognosis” as used herein generally refers to a prediction of the likely course and outcome of a clinical disease. A prognosis of a patient is made by evaluating factors or symptoms of a disease that are indicative of a favorable or unfavorable course or outcome of the disease. The term “prognosis” does not necessarily mean the ability to accurately predict the course or outcome of a disease. The skilled artisan would understand that the term “prognosis” refers to an increased likelihood that a particular course or outcome would occur in a patient exhibiting a given disease, when compared to those individuals not having the disease.

The term “expression” or “expressed” as used in reference to an enzyme or protein means the presence of the enzyme or protein or as used in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “effective amount” or “therapeutically effective amount” is meant the amount of an agent or therapeutic required to ameliorate the disease or symptoms of a disease in a subject suffering from such disease relative to an untreated subject suffering from such disease. The effective amount of active compound(s) used to practice the present disclosure for therapeutic treatment of a disease varies depending upon the manner of administration, gender, age, body weight, and general health of the subject. Ultimately, the attending practitioner, physician or veterinarian will decide the appropriate amount and dosage regimen. In some embodiments, the agents are administered in an effective amount for the treatment or prophylaxis of a disease, disorder, or condition associated with a psychiatric illness as described here. Such amount is referred to as an “effective” amount.

The disclosure provides a number of targets that are useful for the development of highly specific drugs to treat or a disorder characterized by the methods delineated herein. In addition, the methods of the disclosure provide a facile means to identify therapies that are safe and effective for use in subjects. In addition, the methods of the disclosure provide a route for analyzing virtually any number of active agents for effects on a disease described herein with high-volume throughput, high sensitivity, and low complexity.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “high-throughput” is meant a technique that allows for large amounts of proteins or nucleic acids to be tested. For example, high-throughput screening of samples for neurosteroidogenesis enzymes as described here.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent, or “obtaining a sample” means receiving a patient or a control sample directly from a subject or acquiring the sample from another party.

“Primer set” means a set of oligonucleotides (forward and reverse primers) that may be used, for example, for PCR, including but not limited to RT-PCR, qPCR, and qRT-PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition or a standard of comparison. For example, the marker level(s) present in a patient sample may be compared to the level of the marker in a corresponding healthy cell or tissue or in a diseased cell or tissue (e.g., a cell or tissue derived from a subject having endometriosis). In particular embodiments, the polypeptide level present in a patient sample may be compared to the level of said polypeptide present in a corresponding sample obtained at an earlier time point (i.e., prior to treatment), to a cell or tissue of another benign condition. As used herein, the term “sample” includes a biologic sample such as any tissue, cell, fluid, or other material derived from an organism.

By “specifically binds” is meant a compound or antibody that recognizes and binds a polypeptide or polynucleotide of the disclosure, but which does not substantially recognize and bind other molecules in a sample, for example, a biological sample, which naturally includes a polypeptide of the disclosure.

By “subject”, “individual”, and “patient”, used interchangeably here, is meant a mammal, including, but not limited to, a human or non-human mammal, such as a bovine, equine, canine, ovine, or feline for whom diagnosis, prognosis, treatment, or therapy is desired.

A “sample” or “biological sample” as used here refers to a sample of tissue, cells, or fluid isolated or obtained from a subject, including but not limited to, for example, blood, buffy coat, plasma, serum, blood cells (e.g., peripheral blood mononucleated cells (PBMCS)), fecal matter, urine, bone marrow, bile, spinal fluid, lymph fluid, samples of the skin, external secretions of the skin, respiratory, intestinal, and genitourinary tracts, tears, saliva, milk, organs, biopsies and also samples of in vitro cell culture constituents, including, but not limited to, conditioned media resulting from the growth of cells and tissues in culture medium, e.g., recombinant cells, and cell components.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith, such as but not limited to psychiatric illnesses. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

By “vector” is meant a nucleic acid molecule, for example, a plasmid, cosmid, virus, or bacteriophage that is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

Unless specifically stated or obvious from context, as used herein, the term “about” is understood as within a range of normal tolerance in the art, for example, within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 shows a neurosteroidogenic pathway, where allopregnanolone (5) can be synthesized in the brain de novo from cholesterol (1) or by metabolism of the steroid hormone precursor, progesterone (3). The rate limiting step in the synthesis of allopregananolone is the enzyme, 5α-reductase (iii). Other intermediaries include pregnenolone (2) and 5α-dihydroprogesterone (4), with enzymes (i) p450scc, 3β-HSD (ii), and 3α-HSD (iv).

FIGS. 2A-2C demonstrates that manipulation of 5α-reductase is sufficient to alter behavior in mice. FIG. 2A illustrates the behavioral results of a tail suspension test. FIG. 2B shows a reduction in the expression of 5α-reductase 1 and 2 (Srd5a1 and Srd5a2, respectively). FIG. 2C demonstrates avoidance behaviors in a light/dark box test.

FIGS. 3A-3D show that chronic unpredictable stress (CUS) induces decreased 5α-reductase expression and persistent behavioral deficits in mice that are restored with allopregnanolone treatment. FIG. 3A demonstrates that mice subjected to chronic unpredictable stress (CUS) had decreased 5α-reductase expression in the basolateral amygdala (BLA) and behavioral changes (FIGS. 3B-3D). In each graph, from left to right, the first bar is control, second bar is mice following CUS, and the third bar is mice following CUS and with exogenous allopregnanolone treatment.

FIGS. 4A-4D show quantification of 5α-reductase expression at the protein and transcript level in human blood samples. FIG. 4A provides representative Western blot analysis of 5α-reductase type 1 and 2 expression and qRT-PCR measurements of 5α-reductase type 1 and type 2 transcripts, SRD5A1 and SRD5A2, respectively in human whole blood samples (FIGS. 4B-4C). FIG. 4B discloses Forward Primers: SRD5A1 (SEQ ID NO: 1); SRD5A2 (SEQ ID NO: 5); GAPDH (SEQ ID NO: 23) and Reverse Primers: SRD5A1 (SEQ ID NO: 25); SRD5A2 (SEQ ID NO: 26); GAPDH (SEQ ID NO: 27). FIG. 4D illustrates the quantification of expression levels of SRD5A1 (center) and SRD5A2 (right) compared to GAPDH (left).

FIG. 5 illustrates the relationship between 5α-reductase expression and endogenous neurosteroidogenesis.

FIGS. 6A-6C demonstrate different computational approaches for identifying subpopulations of subjects with a reduced capacity for endogenous neurosteroidogenesis.

FIG. 7 provides exemplary computational approaches for correlating 5α-reductase expression with adverse childhood experiences (ACEs). The size of the different clusters of 5α-reductase expression relative to the number of ACEs indicated at the top are shown. The size of the cluster with low 5α-reductase expression (circled dots) is larger with a higher number of ACEs compared to lower number of ACEs or individuals with normal/high levels of 5α-reductase (dots uncircled).

FIGS. 8A-8B show exemplary computational approaches for examining the ability of the biomarker to indicate treatment response. The analyses are based on the relationship between the expression of 5α-reductase and treatment response to NAS-based treatments (FIG. 8A) compared to serotonin reuptake inhibitors (SSRIs) (FIG. 8B). Individuals with a low level of 5α-reductase (pink cluster) will exhibit a bigger reduction in HAM-D scores in response to neurosteroid-based treatments (left panel) compared to individuals with normal/high levels of 5α-reductase (grey cluster).

FIGS. 9A-9B demonstrate the quality and efficiency metrics of the biomarker assay. FIG. 9A shows that inter- and intra-assay variability is very low for the qRT-PCR measurements of GAPDH, SRD5A1, and SRD5A2. FIG. 9B provides a comparison to population samples, where the inter- and intra-assay variability is negligible and does not interfere with the GAPDH, SRD5A1, and SRD5A2 measurements from the patient samples. The ΔCt (inter-intra) mean values on the left side of the graph is presented from left to right as SRD5A1, SRD5A2, and GAPDH, respectively. The peaks on the right side are presented from left to right as GAPDH, SRD5A1, and SRD5A2, respectively.

FIGS. 10A-10B illustrate the stratification of individuals based on SRD5A1 expression. The distribution of SRD5A1 (FIG. 10A) and SRD5A2 (FIG. 10B) expression is used to stratify patients and identify individuals with a reduced capacity for endogenous neurosteroid synthesis (low expression).

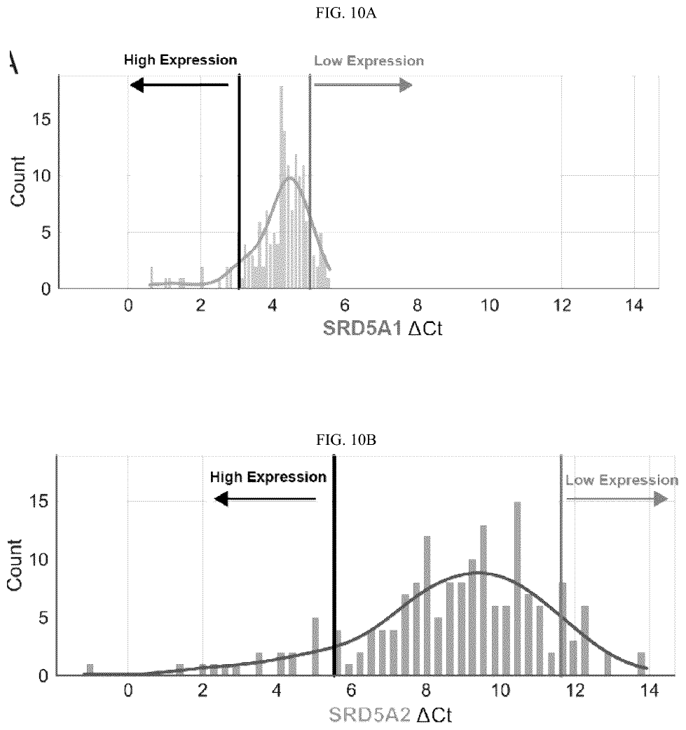

FIGS. 11A-11B show individuals with lower SRD5A expression as having more severe anxiety symptoms. Individuals with a reduced capacity for endogenous neurosteroid synthesis based on total SRD5A expression (right bars for each graph and sex at birth group) have increased STATE (FIG. 11A) and TRAIT (FIG. 11B) anxiety scores compared to the general population (left bars for each graph and sex at birth). * denotes p<0.05.

FIGS. 12A-12B illustrate individuals with higher SRD5A expression as having more severe PTSD symptoms. Low SRD5A expression is associated with lower CAPs scores: total CAPS scores (FIG. 12A) and CAPS_e symptom subscale scores (FIG. 12B) related to hyperarousal (e.g., hypervigilance, startling easily). * denotes p<0.05.

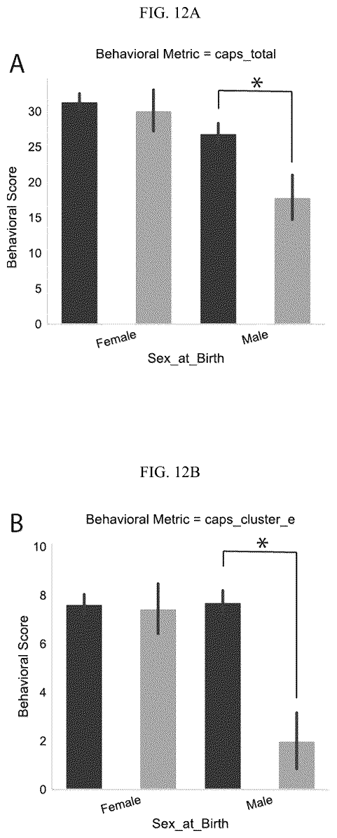

### DETAILED DESCRIPTION

The disclosure provides compositions and methods for characterizing psychiatric illness (e.g., depression) associated with neurosteroidogenic pathways, and therapy selection.

The disclosure is based, at least in part, on the discovery of deficits in the neurosteroidogenic pathway that contribute to the pathophysiology of psychiatric illnesses, including but not limited to, depression (e.g., postpartum depression, peripartum depression, perimenopausal depression, major depression; infertility-related depression); anxiety disorders (e.g., generalized anxiety disorder; panic disorder; social anxiety disorder; phobia-related disorder (e.g., specific or simple phobias; agoraphobia; separation anxiety disorder; selective mutism); post-traumatic stress disorder (PTSD); premenstrual dysphoric disorder (PMDD); traumatic brain injury; concussion; epilepsy (e.g., catamenial epilepsy), in a subset patient population. In particular, expression of neurosteroidogenesis enzymes, such as 5α-reductase, in patient samples correlate to the capacity for endogenous neurosteroidogenesis. Furthermore, endogenous neurosteroids were found to contribute to a baseline affective state which is disrupted by risk factors for psychiatric illness, such as chronic stress. Since a large patient population is non-responsive to standard treatments, the biomarkers and methods described here identify subsets or populations of patients having impaired endogenous neurosteroidogenesis, where the biomarkers serve as predictive biomarkers for identifying patients having a psychiatric illness that is responsive to neuroactive steroid (NAS)-based treatments.

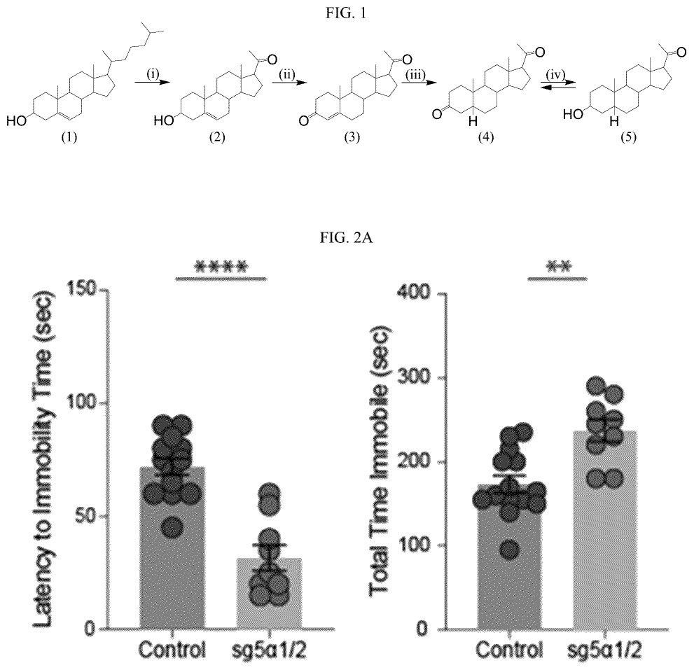

Independent of origin, neuroactive steroids (NAS) are steroids that exert actions and are synthesized locally in the central nervous system (CNS). NAS, such as allopregnanolone (ALLO) and tetrahydrodeoxycorticosterone (THDOC), can exogenously treat depression and seizures. However, NAS can be synthesized in the brain, either by metabolizing steroid hormone precursors or de novo from cholesterol. Neurosteroid synthesis, or neurosteroidogenesis, involves the sequential action of steroidogenic enzymes, including cytochrome P450 side-chain cleavage enzyme (P450scc) (i), 3β-hydroxysteroid dehydrogenase (ii), 5α-reductase (iii), and 3α-hydroxysteroid dehydrogenase (iv), from cholesterol (1) to pregnenolone (2) to progesterone (3) to 5α-dihydroprogesterone (4) to allopregnanolone (5) (FIG. 1).

### Biomarkers and Kits

In some embodiments of the disclosure, a panel of one or more biomarkers of endogenous neurosteroidogenesis is provided. The biomarkers described here include neuroactive steroids, metabolites, intermediaries, and neurosteroidogenic enzymes in neurosteroidogenic pathways, including but not limited to those from cholesterol to allopregnanolone and those involving the P450 side chain cleavage (P450scc), 3β hydroxysteroid dehydrogenase (3β-HSD), or 3α-hydroxysteroid dehydrogenase (3α-HSD). Non-limiting examples of such biomarkers include: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKRIC2); aldo-keto reductase family 1, member C3 (AKRIC3); aldo-keto reductase family 1, member C4 (AKR1C4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

The panel of one or more biomarkers described here can further include reference controls, such as housekeeping proteins or housekeeping genes. Some embodiments provide for the panel of one or more biomarkers, where the one or more biomarkers are on a substrate (e.g., plate, slide, bead, pellet, disk, particle, microparticle, nanoparticle, strand, precipitate, optionally porous gel, sheets, tube, sphere, container, capillary, pad, slice, film, chip, multi-well plate or dish, optical fiber, etc.)

Various embodiments of the disclosure provide for biomarkers that are useful in detecting or measuring expression of such biomarkers in a subject or sample from the subject. The biomarkers described here are useful in identifying subjects having or at risk of developing a psychiatric illness, identifying those subject responsive to available neurosteroid-based treatments, identifying those subjects responsive to novel treatments targeting endogenous neurosteroidogenesis, and in measuring efficacy of such treatments.

Further embodiments of the disclosure provide kits for: diagnosing a psychiatric illness, determining risk of developing a psychiatric illness, identifying responsiveness to new or existing neurosteroid-based treatments, and measuring efficacy, in a subject, wherein the kits can be used to detect the biomarkers of the present disclosure. For example, the kits can be used to detect any one or more of the biomarkers described herein, which are differentially expressed in samples of a subject or individual having or at risk of having a psychiatric illness and a healthy or non-affected subject who does not suffer from a psychiatric illness. The kit can include one or more reagents for measuring the levels of expression of biomarkers of endogenous neurosteroidogenesis, including but not limited to, neuroactive steroids, metabolites, intermediaries, and neurosteroidogenic enzymes, a substrate or container for holding a biological sample isolated from a human subject suspected of having a psychiatric illness; and printed instructions for reacting reagents with the biological sample or a portion of the biological sample for measuring the levels of expression of the biomarkers or test control in the biological sample. The reagents can be packaged in separate containers. The kits can further comprise one or more control reference samples and reagents for performing an immunoassay, PCR, or microarray screening and/or analyses.

In one embodiment, the kit comprises reagents for measuring the levels of expression of biomarkers selected from the group consisting of: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKRIC4); and hydroxy-delta-5-steroid dehydrogenase, 3 beta- and steroid delta-isomerase 2 (HSD3B2) for distinguishing samples from individuals having or at risk of having a psychiatric illness from healthy individuals who do not have a psychiatric illness.

Additional embodiments of the disclosure provide for the kit further comprising an array or microarray for analyzing a plurality of biomarker proteins or polynucleotides in a sample. In one aspect, the array comprises an antibody against or binds to any of the described biomarkers. Another aspect provides the array, such as a droplet array, comprising a platform for analyzing a plurality of biomarker proteins or polynucleotides in a sample, where biomarker-specific primer sets are used for the analysis. For example, a substrate such as a multi-well plate or silicon chip, can be used to generate a droplet array containing cells or cell suspension for performing RT-PCR, where the cells are lysed, undergo reverse transcription, where a reverse transcription mixture is added to the cells, a PCR mixture subsequently added, and the array undergoes RT-PCR to quantify the biomarker gene expression levels.

Non-limiting examples of biomarker-specific primers or primer sets used for the analysis or methods described here include:

SRD5A1:
Forward
(SEQ ID NO: 1)
CAGACCCTGTCTGCTGTTATC
Reverse
(SEQ ID NO: 2)
GATAACAGCAGACAGGGTCTG
Forward
(SEQ ID NO: 3)
GAGATATTCAGCTGAGACCC
Reverse
(SEQ ID NO: 4)
TTAGTATGTGGGCAGCTTGG
SRD5A2:
Forward
(SEQ ID NO: 5)
CCCTTTGAGGGTCAGATGTATG
Reverse
(SEQ ID NO: 6)
CATACATCTGACCCTCAAAGGG
Forward
(SEQ ID NO: 7)
ATTTGTGTGGCAGAGAGAGG
Reverse
(SEQ ID NO: 8)
TTGATTGACTGCCTGGATGG
TSPO:
Forward
(SEQ ID NO: 9)
TCCTACCTGGTCTGGAAAGAG
Reverse
(SEQ ID NO: 10)
CTCTTTCCAGACCAGGTAGGA
CYP11A1:
Forward
(SEQ ID NO: 11)
CCAGAAGTATGGCCCGATTTA
Reverse
(SEQ ID NO: 12)
TAAATCGGGCCATACTTCTGG
AKR1C1:
Forward
(SEQ ID NO: 13)
GGGCAGGTGACAGGTATTTAT
Reverse
(SEQ ID NO: 14)
ATAAATACCTGTCACCTGCCC
AKR1C2:
Forward
(SEQ ID NO: 15)
CGGGTTCCACCATATTGATTCT
Reverse
(SEQ ID NO: 16)
AGAATCAATATGGTGGAACCCG
AKR1C3:
Forward
(SEQ ID NO: 17)
CCAGAGGTTCCGAGAAGTAAAG
Reverse
(SEQ ID NO: 18)
CTTTACTTCTCGGAACCTCTGG
AKR1C4:
Forward
(SEQ ID NO: 19)
GGAGGTCATGGAGAAGTGTAAG
Reverse
(SEQ ID NO: 20)
CTTACACTTCTCCATGACCTCC
HSD3B2:
Forward
(SEQ ID NO: 21)
ACTCAGCCCAATTTACTCCTATC
Reverse
(SEQ ID NO: 22)
GATAGGAGTAAATTGGGCTGAGT.

An exemplary test control-specific primer or primer set includes those for the housekeeping gene, GAPDH: Forward: CGG AGT CAA CGG ATT TGG TCG TAT (SEQ ID NO: 23); Reverse: ATA CGA CCA AAT CCG TTG ACT CCG (SEQ ID NO: 24). One of ordinary skill in the art has common knowledge in the field to design, synthesize, or use primers directed to any of the biomarkers described here, their family members, or isomers thereof, where such primers are useful in the compositions, methods, and systems of the disclosure.

In further embodiments, a kit comprises detection means of such biomarkers described here including additional biomarkers of endogenous neurosteroidogenesis. For example, the kit comprises antibodies against any of the one or more biomarkers of endogenous neurosteroidogenesis, such as those selected from the group consisting of: steroid 5 alpha-reductase 1 (SRD5A1); steroid 5 alpha reductase 2 (SRD5A2); translocator protein (TSPO); Glyceraldehyde 3-phosphate dehydrogenase (GAPDH); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKR1C4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

Other embodiments are directed to kits comprising a primer set and a detectably labeled (e.g., fluorescent) probe that specifically amplifies and detects at least one or more biomarkers of endogenous neurosteroidogenesis, such as those selected from the group consisting of: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKR1C3); aldo-keto reductase family 1, member C4 (AKR1C4); and 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

In some embodiments, the kits described here can further comprise an antibody against a test control protein, where the protein is found in all subjects. Such a test control protein can be a housekeeping protein, for example, β-actin or glyceraldehyde-3-phosphate dehydrogenase (GAPDH). Other embodiments provide for kits further comprising a primer set and a detectably labeled probe that specifically amplifies and detects a test control gene product, wherein the detectably labeled probe that specifically detects the test control gene product is differently labeled than the detectably labeled probes that specifically amplify and detect the at least one or more biomarkers in the kits. Some aspects of the kits described here include detectably labeled probes that are all labeled with the same detection label. additional aspects of the described kits further comprise reverse transcriptase and/or thermostable DNA polymerase; or an enzyme with both reverse transcriptase and DNA polymerase activity. The kit, in some embodiments, comprises one or more containers for reagents contained in the kit. Non-limiting examples of reagents include reverse transcriptase, DNA polymerase, deoxynucleotide triphosphates (dNTPs), probes, primers, positive/negative controls or control samples, buffers, and the like. Suitable containers include, for example, bottles, vials, syringes, and test tubes. Containers can be formed from a variety of materials, including glass or plastic. The kit can also comprise a package insert containing written instructions for methods of diagnosing psychiatric illnesses, determining risk of developing psychiatric illnesses, identifying responsiveness to new or existing neurosteroid-based treatments, and measuring efficacy of such treatments.

### Methods

In some embodiments of the disclosure, biomarkers in a sample are detectable and quantifiable using any suitable method known in the art. Direct or indirect biomarker expression measurement can be achieved using such methods. For example, expression levels of RNAs or proteins of biomarkers can be directly quantified. The amount of a biomarker can also be determined indirectly by measuring levels of cDNAs, amplified RNAs or DNAs, or by measuring quantities or activities of RNAs, proteins, metabolites, or other molecules indicative of the expression level of the one or more biomarkers described here. Methods for measuring biomarkers in a sample have several applications, such as for example, one or more biomarkers described here can be measured to characterize the level of a polypeptide that functions in neurosteroidogenesis, for the diagnosis of a psychiatric disease, determination of the appropriate treatment for a subject, efficacy of or responses in a subject to treatment, or identifying therapeutic agents that modulate expression of the biomarkers in vivo or in vitro.

Some embodiments are directed to determining the expression levels of the biomarkers described here by measuring protein and/or polynucleotide levels of such biomarkers. In one embodiment of the disclosure, provided here is a method of identifying a subject responsive to treatment of a psychiatric illness in a subject, the method comprising: (a) measuring levels of expression of one or more biomarkers in a sample from the subject compared to a reference from a control indicative that the subject suffers from or is at risk of a psychiatric illness; and (b) treating the subject for the psychiatric illness if the subject has a positive diagnosis for responding to treatment of the psychiatric illness. The one or more biomarkers measured are in a panel of biomarkers, or the method measures the levels of expression of the one or more biomarkers, where the biomarkers of endogenous neurosteroidogenesis are such as those selected from the group consisting of: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKRIC4); 3β-hydroxysteroid dehydrogenase (e.g., hydroxy-delta-5-steroid dehydrogenase, 3 beta- and steroid delta-isomerase 2 (HSD3B2)); and Steroidogenic acute regulatory protein (StAR). In some aspects, if the expression level in the subject sample is at least the same as the levels of expression in the positive control, the subject has a positive diagnosis for the psychiatric illness and/or for responding to a neuroactive steroid treatment described here. Such methods of detecting expression of the one or more biomarkers comprise, in some embodiments, contacting a sample from a subject with an antibody, or fragment thereof, against one or more biomarkers described here, where the contacting occurs under conditions allowing for binding of the antibody, or fragment thereof, to the biomarker, and detecting a complex formed by the antibody and the biomarker in the sample, using a detectable label.

In other embodiments, the expression levels of the one or more biomarkers disclosed here are established by measuring polynucleotide levels of the biomarkers. The transcript levels of specific biomarker genes are determined from the amount of mRNA or polynucleotides derived therefrom that are present in a sample from a subject having or suspected of having a psychiatric illness. Polynucleotides are detected and quantified by methods, including but not limited to, polymerase chain reaction (PCR), reverse transcriptase polymerase chain reaction (RT-PCR), serial analysis of gene expression (SAGE), Northern blot, solid-state nanopore detection, and other methods commonly known and used in the art for detecting and quantifying gene transcripts.

Some embodiments are directed to methods of detecting or measuring expression by determining the level expression in a manner that is qualitative, semi-quantitative, or quantitative. Such methods occur by detection of proteins or nucleic acid molecules (e.g., DNA, RNA, and/or mRNA). Non-limiting methods include analysis by microarray, PCR (such as RT-PCR, real-time PCR, or qRT-PCR), Northern blot, Western blot, ELISA, and mass spectrometry.

This disclosure contemplates traditional immunoassays including, for example, Western blot, sandwich immunoassays including ELISA and other enzyme immunoassays, fluorescence-based immunoassays, and chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In aspects of the disclosure, a sample is analyzed by means of a biochip (also known as a microarray). The polypeptides and nucleic acid molecules of the disclosure are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In aspects of the disclosure, a sample is analyzed by means of a protein biochip (also known as a protein microarray). Such biochips are useful in high-throughput low-cost screens to identify alterations in the expression or post-translation modification of a polypeptide of the disclosure, or a fragment thereof. In embodiments, a protein biochip of the disclosure binds a biomarker present in a subject sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the disclosure) are spotted on a substrate using any convenient method known to the skilled artisan (e.g., by hand or by inkjet printer).

In embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, CA), Zyomyx (Hayward, CA), Packard BioScience Company (Meriden, CT), Phylos (Lexington, MA), Invitrogen (Carlsbad, CA), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the disclosure, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/25116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g., RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In other embodiments, wash steps will occur at 68° C. in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

In various embodiments, the expression level of a biomarker such as an mRNA is measured by a technique selected from the group consisting of quantitative real-time PCR (qPCR), reverse transcription polymerase chain reaction (RT-PCR), rapid amplification of cDNA ends (RACE-PCR), multiplex RT-PCR, Northern blotting, nuclease protection assays, in situ hybridization, serial analysis of gene expression (SAGE), RNA microarray, RNA microarray and gene chips, and RNA sequencing (RNA-seq). These techniques are commonly known and used, and can be used in measuring the expression level of a biomarker polynucleotide.

A variation of RT-PCR is real time quantitative RT-PCR (qRT-PCR), which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR. Related probes and quantitative amplification procedures, instruments for executing quantitative PCR in microtiter plates are commercially available.

An expression profile can be derived from a subject prior to or subsequent to a diagnosis of a psychiatric illness, from a biological sample collected from a subject at one or more time points prior to or following treatment or therapy, from a biological sample collected from a subject at one or more time points during which there is no treatment or therapy (e.g., to monitor progression of the psychiatric illness or to assess development of the psychiatric illness in a subject diagnosed with or at risk for a psychiatric illness), or from a sample collected from a healthy subject who does not suffer from a psychiatric illness. Such an “expression profile” comprises one or more values corresponding to a measurement of the relative abundance, level, presence, or absence of expression of a discriminative gene, messenger RNA (mRNA), microRNA (miRNA), or long non-coding RNA (IncRNA), such as those of the one or more biomarkers described here.

In hybridization or amplification assays, isolated mRNA can be used. Such non-limiting assays include, Southern or Northern analyses, PCR analyses and probe arrays, such as NanoString Assays. One exemplary method for the detection of biomarker mRNA levels comprises contacting the isolated mRNA or synthesized cDNA with a nucleic acid molecule (such as a probe) that can hybridize to the mRNA encoded by the biomarker gene being detected. The nucleic acid probe can be, for example, a cDNA, or a portion thereof, such as an oligonucleotide of at least 10, 15, 20, 30, 50, 100, 250, or 500 nucleotides in length and sufficient to specifically hybridize under stringent conditions to the non-natural cDNA or mRNA biomarker described here.

Various embodiments provide for mRNA obtained from a sample that is converted to complementary DNA (cDNA) in a hybridization reaction. The cDNA can be amplified by, for example, the polymerase chain reaction (PCR) or another amplification method known in the art. The amplification process can create hundreds of millions of copies of cDNA for each individual cDNA molecule in the initial material. cDNA products can be detected by real-time polymerase chain reaction (PCR) through the fluorescent probes that hybridize with the cDNA products. For example, in one embodiment, biomarker detection is determined by detectable label or fluorescent quantitative RT-PCR (e.g., detectable probes).

Several embodiments of the disclosure can incorporate any of the aforementioned detection means (i.e., detection by biochip, protein biochip, nucleic acid biochip) to perform any of the methods of the disclosure described here. In some embodiments, provided are methods of diagnosing and treating a psychiatric illness in a subject, where the method comprises: (a) measuring levels of expression of one or more biomarkers in a sample from the subject compared to a reference from a control indicative that the subject suffers from or is at risk of a psychiatric illness. The one or more biomarkers of such methods are selected from the group consisting of: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKRIC4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR). If the levels of expression in the sample of the subject is at least the same as the levels of expression in the positive control for the psychiatric illness, then the sample of the subject has a positive diagnosis for the psychiatric illness. Accordingly, the subject is then treated for the psychiatric illness if the subject has a positive diagnosis for the psychiatric illness.

Another embodiment of the disclosure provides a method of determining the efficacy of a medicament or therapeutic agent for treating a psychiatric illness in a subject. In some aspects, the method comprises: (a) measuring levels of expression of the described one or more biomarkers in a first sample from the subject exposed to the medicament or therapeutic agent. Such biomarkers are selected from the group consisting of: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKR1C4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR). The method further comprises (b) measuring levels of expression of the one or more biomarkers in a second sample from the subject not exposed to the medicament or therapeutic agent. The method is directed to comparing the levels of expression of the one or more biomarkers in the first sample and the second sample, where an increased level of expression of the one or more biomarkers in the first sample relative to the second sample indicates that the medicament or therapeutic agent is efficacious for treating the psychiatric illness in the subject; and then (c) continue treating the subject for the psychiatric illness if the levels of expression in the first sample is greater than the levels expression in the second sample. In some aspects, the measuring comprises: isolating total protein from the subject sample (or the sample from the subject) and from the sample from the reference control (or positive control for the psychiatric illness or negative control from a healthy subject or an individual who is not suffering from a psychiatric illness); applying antibodies, or fragments thereof, against any of the one or more biomarkers and against a test control (e.g., a protein found in all samples, such as a housekeeping protein, β-actin, GAPDH); and quantifying protein expression through a detectable label, for example. Other aspects are directed to methods where measuring occurs by isolating RNA from the subject sample and from the reference control sample; adding biomarker-specific primers and test control-specific primers (e.g., to housekeeping genes, β-actin; GAPDH); and quantifying levels of expression through a detectable label.

In further embodiments of the methods described here, treating comprises administering a medicament or therapeutic agent that increases the levels of endogenous neuroactive steroids, metabolites, intermediaries, and neurosteroidogenic enzymes involved in endogenous neurosteroidogenesis. The medicament or therapeutic agent for use in treating a subject selected as having a reduced capacity for neurosteroidogenesis includes administration of either exogenous neurosteroids, metabolites, or intermediaries or an agent that is an activator or inducer of or increases endogenous neurosteroidogenesis. Non-limiting examples of such medicaments or therapeutic agents include: treatments targeting or using steroid 5 alpha-reductase 1 (SRD5A1); steroid 5 alpha-reductase 2 (SRD5A2); translocator protein (TSPO); cytochrome P450 family 11 subfamily A member 1 (CYP11A1); aldo-keto reductase 1C superfamily (e.g., ALRIC1, AKRIC2, AKRIC3, AKRIC4); 3β-hydroxysteroid dehydrogenase (3-β-HSD (e.g., hydroxy-delta-5-steroid dehydrogenase, 3-beta- and steroid deltaisomerase 2 (HSD3B2)); Steroidogenic acute regulatory protein (StAR); or positive allosteric modulator of GABAA receptors (e.g., ganaxolone (ZTALMY®); brexanolone (ZULRESSOR); zuranolone (ZURZUVAE™)); progesterone analogue (e.g., (8S,9S,10R,13S,14S,17S)-17-((E)-1-(Hydroxyimino)ethyl)-10,13-dimethyl-6,7,8,9,10,11,12,13,14,15,16,17-dodecahydro-1H-cyclopenta[a]phenanthren-3 (2H)-one (EIDD-036)); allopregnanolone prodrug (e.g., LYT-300); brexanolone prodrug (e.g., ester prodrugs, carbonate prodrugs); and delta-subunit-selective GABAA receptor modulator (e.g., DS2); and/or activators or inducers of such neuroactive steroids, metabolites, intermediaries, and neurosteroidogenic enzymes.

Some embodiments described here include a method of treating a subject having a psychiatric illness characterized by reduced levels of one or more biomarkers of endogenous neurosteroidogenesis. The method comprises: (a) detecting expression of the one or more biomarkers in a sample directly or indirectly obtained from the subject by contacting the sample with an antibody against the one or more biomarkers and determining if the antibody bound to the one or more biomarkers, or alternatively, contacting the sample with a primer set to the one or more biomarkers and detecting expression of the one or more biomarkers; (b) comparing expression of the one or more biomarkers in the sample to a reference from a healthy control subject who does not suffer from a psychiatric illness; and (c) administering a neuroactive steroid (NAS)-based agent or therapeutic to the subject if the sample has reduced levels of one or more biomarkers of endogenous neurosteroidogenesis as compared to the reference value. Such biomarkers include one or more selected from the group consisting of: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKR1C1); aldo-keto reductase family 1, member C2 (AKR1C2); aldo-keto reductase family 1, member C3 (AKR1C3); aldo-keto reductase family 1, member C4 (AKRIC4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR). The NAS-based treatment, agent, or therapeutic includes, but is not limited to, treatments targeting or using steroid 5 alpha-reductase 1 (SRD5A1); steroid 5 alpha-reductase 2 (SRD5A2); translocator protein (TSPO); cytochrome P450 family 11 subfamily A member 1 (CYP11A1); aldo-keto reductase 1C superfamily (e.g., ALRIC1, AKRIC2, AKRIC3, AKRIC4); 3β-hydroxysteroid dehydrogenase (3-β-HSD (e.g., hydroxy-delta-5-steroid dehydrogenase, 3-beta- and steroid deltaisomerase 2 (HSD3B2); Steroidogenic acute regulatory protein (StAR); or positive allosteric modulator of GABAA receptors (e.g., ganaxolone (ZTALMY®); brexanolone (ZULRESSOR); zuranolone (ZURZUVAE™)); progesterone analogue (e.g., (8S,9S,10R, 13S,14S,17S)-17-((E)-1-(Hydroxyimino)ethyl)-10,13-dimethyl-6,7,8,9,10,11,12,13,14,15,16,17-dodecahydro-1H-cyclopenta[a]phenanthren-3 (2H)-one (EIDD-036)); allopregnanolone prodrug (e.g., LYT-300); brexanolone prodrug (e.g., ester prodrugs, carbonate prodrugs); and delta-subunit-selective GABAA receptor modulator (e.g., DS2); and/or activators or inducers of such neuroactive steroids, metabolites, intermediaries, and neurosteroidogenic enzymes. Further embodiments are directed to such methods where the psychiatric illness is selected from the group consisting of: depression; anxiety disorders; post-traumatic stress disorder (PTSD); premenstrual dysphoric disorder (PMDD); traumatic brain injury; concussion; epilepsy. In particular embodiments, the psychiatric illness is selected from the group consisting of: postpartum depression, peripartum depression, perimenopausal depression, major depression; infertility-related depression; generalized anxiety disorder; panic disorder; social anxiety disorder; specific or simple phobias; agoraphobia; separation anxiety disorder; selective mutism; catamenial epilepsy.

The biomarkers and methods described here are for diagnosing, determining risk of, prognosing, stratifying patients having, or determining the efficacy NAS-based treatments of a psychiatric illness. In some aspects, the psychiatric illness or indications applicable to the biomarkers and methods of the disclosure are selected from the group consisting of: depression; anxiety disorders; post-traumatic stress disorder (PTSD); premenstrual dysphoric disorder (PMDD); traumatic brain injury; concussion; epilepsy. Some of the psychiatric illnesses more particularly include those selected from the group consisting of: postpartum depression, peripartum depression, perimenopausal depression, major depression; infertility-related depression; generalized anxiety disorder; panic disorder; social anxiety disorder; specific or simple phobias; agoraphobia; separation anxiety disorder; selective mutism; catamenial epilepsy.

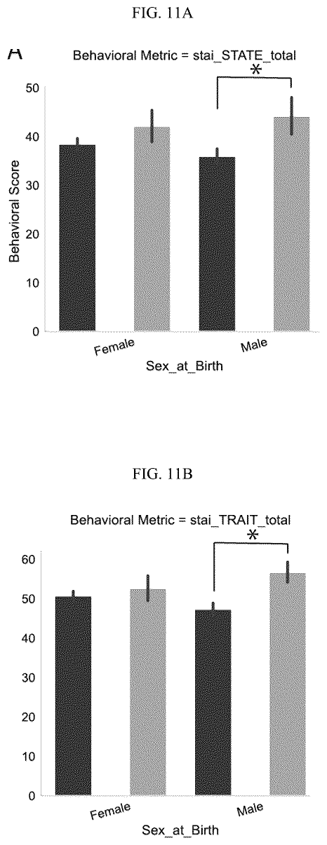

In some embodiments biomarkers and their expression can be used to diagnose, determine risk of, prognose, stratify patients having, determine the efficacy of NAS-based treatment of a psychiatric illness described here, or any combinations of the aforementioned. Additional embodiments are directed to gender-based stratification of patients associated with symptoms of any of the psychiatric illnesses described here based on biomarker expression. In one aspect, biomarker expression associated with any one of the psychiatric illnesses described here can be associated with only males. Another aspect is directed to biomarker expression associated with any one of the psychiatric illnesses described that are associated with only females. In further aspects, biomarker expression associated with any one of the psychiatric illnesses described here can be associated with both males and females. For example, FIGS. 11A-11B demonstrate that lower SRD5A expression in males are associated with more severe anxiety symptoms. While higher SRD5A expression in males are associated with more severe PTSD symptoms.

Provided in some of the method embodiments of the disclosure are biomarker-specific primers for the detection and quantification of such biomarkers in a sample, where the biomarker-specific primers or primer sets are selected from the group consisting of:

SRD5A1:
Forward
(SEQ ID NO: 1)
CAGACCCTGTCTGCTGTTATC
Reverse
(SEQ ID NO: 2)
GATAACAGCAGACAGGGTCTG
Forward
(SEQ ID NO: 3)
GAGATATTCAGCTGAGACCC
Reverse
(SEQ ID NO: 4)
TTAGTATGTGGGCAGCTTGG
SRD5A2:
Forward
(SEQ ID NO: 5)
CCCTTTGAGGGTCAGATGTATG
Reverse
(SEQ ID NO: 6)
CATACATCTGACCCTCAAAGGG
Forward
(SEQ ID NO: 7)
ATTTGTGTGGCAGAGAGAGG
Reverse
(SEQ ID NO: 8)
TTGATTGACTGCCTGGATGG
TSPO:
Forward
(SEQ ID NO: 9)
TCCTACCTGGTCTGGAAAGAG
Reverse
(SEQ ID NO: 10)
CTCTTTCCAGACCAGGTAGGA
CYP11A1:
Forward
(SEQ ID NO: 11)
CCAGAAGTATGGCCCGATTTA
Reverse
(SEQ ID NO: 12)
TAAATCGGGCCATACTTCTGG
AKR1C1:
Forward
(SEQ ID NO: 13)
GGGCAGGTGACAGGTATTTAT
Reverse
(SEQ ID NO: 14)
ATAAATACCTGTCACCTGCCC
AKR1C2:
Forward
(SEQ ID NO: 15)
CGGGTTCCACCATATTGATTCT
Reverse
(SEQ ID NO: 16)
AGAATCAATATGGTGGAACCCG
AKR1C3:
Forward
(SEQ ID NO: 17)
CCAGAGGTTCCGAGAAGTAAAG
Reverse
(SEQ ID NO: 18)
CTTTACTTCTCGGAACCTCTGG
AKR1C4:
Forward
(SEQ ID NO: 19)
GGAGGTCATGGAGAAGTGTAAG
Reverse
(SEQ ID NO: 20)
CTTACACTTCTCCATGACCTCC
HSD3B2:
Forward
(SEQ ID NO: 21)
ACTCAGCCCAATTTACTCCTATC
Reverse
(SEQ ID NO: 22)
GATAGGAGTAAATTGGGCTGAGT.

In those embodiments where test control-specific primers or primer sets are used, the test control-specific primers or primer sets can be to a housekeeping gene, such as but not limited to β-actin and GAPDH. A non-limiting primer set for the test control includes GAPDH: Forward: CGG AGT CAA CGG ATT TGG TCG TAT (SEQ ID NO: 23); Reverse: ATA CGA CCA AAT CCG TTG ACT CCG (SEQ ID NO: 24). One of ordinary skill in the art has common knowledge in the field to design, synthesize, or use primers directed to any of the biomarkers described here, their family members, or isomers thereof, where such primers are useful in the compositions, methods, and systems of the disclosure.

In any of the embodiments described here, the sample can include whole blood, serum, urine, and a buccal smear.

In any embodiment, the test control can include a housekeeping protein or a housekeeping gene. For example, the test control can include β-actin or glyceraldehyde-3-phosphate dehydrogenase (GAPDH).

### Hardware and Software

In any of the methods described herein, the measurement of the biomarker(s) can be correlated with disease state (e.g., capacity for neurosteroidogenesis, psychiatric disease) and can be performed on general-purpose or specially-programmed hardware or software.

In aspects, the analysis is performed by a software classification algorithm. The analysis of analytes by any detection method well known in the art, including, but not limited to the methods described herein, will generate results that are subject to data processing. Data processing can be performed by the software classification algorithm. Such software classification algorithms are well known in the art and one of ordinary skill can readily select and use the appropriate software to analyze the results obtained from a specific detection method.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data generated by detection of biomarkers can be analyzed with the use of a programmable digital computer. Data analysis can include steps of determining signal strength of a biomarker and removing data deviating from a predetermined statistical distribution.

In aspects, software used to analyze the data can include code that applies an algorithm to the analysis of the results. The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure. Particularly useful techniques for particular embodiments will be discussed in the sections that follow.

In some embodiments, one or more of the concepts, principles, methodologies, devices, or systems described here can be used in conjunction or combination with one or more of the concepts, principles, methodologies, devices, or systems described in the materials included herewith as Appendix A.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

### Example 1: Neurosteroidogenesis

Through the metabolism of steroid hormone precursors originating from the periphery or de novo from cholesterol, neurosteroids are synthesized in the brain. Neurosteroid synthesis, also termed neurosteroidogenesis, involves the sequential action of steroidogenic enzymes, including cytochrome P450 side-chain cleavage enzyme (P450scc; (i)), 3β-hydroxysteroid dehydrogenase (ii), 5α-reductase (iii), and 3α-hydroxysteroid dehydrogenase (iv) (FIG. 1). Associating neuroactive steroid (NAS) levels with depression is challenging since neurosteroid levels are dynamic and provide only a “snapshot” at any given time. Measuring 5α-reductase (iii), the rate-limiting enzyme in the neurosteroidogenic pathway, correlated with endogenous neurosteroidogenesis. (See, e.g., FIG. 1).

Reduced levels of 5α-reductase reflected impaired endogenous neurosteroidogenesis, which enabled stratification of patients with a specific underlying neurobiology of disease and identifying a subset of patients who are responsive to NAS-based treatments, thereby specifically targeting this unique underlying etiology of disease. As described in detail below, 5α-reductase is useful as a biomarker for stratifying patients having or at risk of developing depression.

### Example 2: Behavioral Alterations Following 5α-Reductase Manipulation

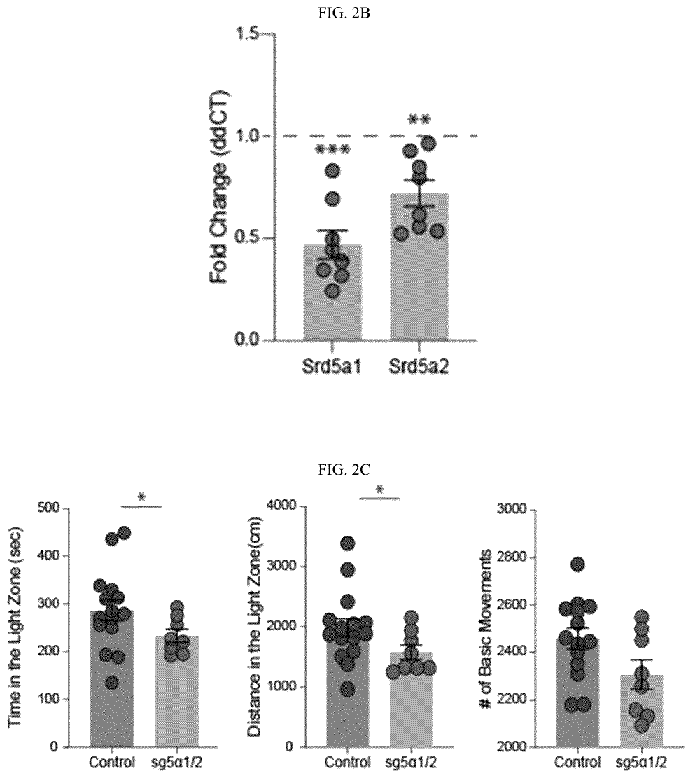

A loss of key enzymes involved in neurosteroid synthesis, 5α-reductase type 1 and 2, induced behavioral deficits in mice (FIGS. 2A-2C). In vivo studies manipulating 5α-reductase 1 and 2 suggested that these are effective targets for altering behavioral outcomes. CRISPR knockdown of 5α-reductase in the basolateral amygdala (BLA) (FIG. 2B) was sufficient to increase learned helplessness (FIG. 2A) and avoidance behaviors in mice (FIG. 2C). CRISPR knockdown of 5α-reductase 1 and 2 was accomplished by injecting sgSrd5a1 and sgSrd5a2 into the BLA of constitutively expressing Cas9 mice. Three weeks following stereotaxic injection of these guide RNAs into the BLA, a reduction in the expression of transcript for 5α-reductase 1 and 2 (Srd5a1 and Srd5a2, respectively) was observed. Mice with a knockdown of 5α-reductase 1 and 2 expression exhibited increased stress-induced helplessness behaviors in the tail suspension test (FIG. 2A) and increased avoidance behaviors in the light/dark box test (FIG. 2C). n=8-15 per group. * denotes p<0.05, **p<0.01, ***p<0.001, ****p<0.0001 using an unpaired t-test.

Using a lentivirus expressing Srd5a1 (Origene Cat No. MR203282) or Srd5a2 (Origene Cat No. MR223814), the genes encoding 5α-reductase type 1 and 2, respectively, 5α-reductase 1 or 2 was overexpressed, which decreased avoidance behaviors and decreased learned helplessness. These data demonstrated that changes in 5α-reductase levels were sufficient to alter behavioral outcomes. Deficits in the expression of these neurosteroidogenic enzymes was associated with depression in a subset of patients.

### Example 3: Behavioral Deficits Associated with Chronic Unpredictable Stress and Decreased 5α-Reductase

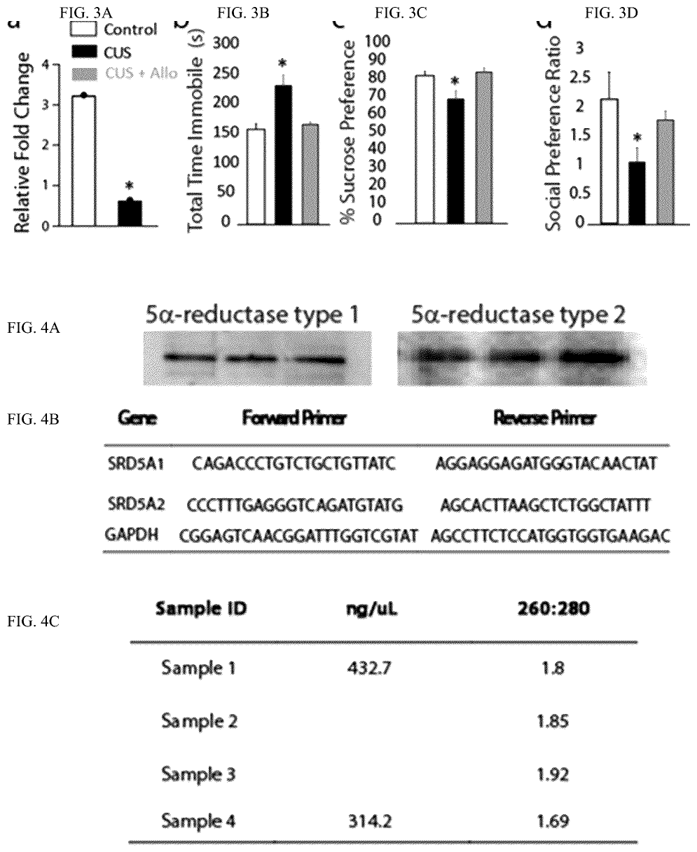

Decreased expression of 5α-reductase 1 and 2 is associated with chronic stress, a major risk factor for depression. Mice subjected to chronic unpredictable stress (CUS), which consists of alternating stressors administered only during the dark period, including cage tilt, unstable cage, soiled cage, difficult access to food, confinement, restraint stress (30 min), and forced swim test (6 min), administered during the dark period for 3 consecutive weeks, exhibited deficits in 5α-reductase expression in the basolateral amygdala (BLA) (FIG. 3A) and behavioral changes, including increased learned helplessness, decreased social interaction, and increased anhedonia (FIGS. 3B-3D). Exogenous allopregnanolone treatment restored the behavioral state of the mice following CUS (FIGS. 3B-3D). Reduced endogenous neurosteroids impaired the affective state. These results indicate that measuring levels of 5α-reductase is useful for stratifying patients into treatment groups to select patients that are responsive to NAS-based treatments.

### Example 4: Methods of Measuring 5α-Reductase Expression

Blood samples, stored at −80° C., were obtained from study participants diagnosed with postpartum depression (PPD), perimenopausal depression (PMD), major depressive disorder (MDD), and individuals without a psychiatric diagnosis (controls), collected as part of previous research involving the mass psychogenic illnesses (MPIs). Peripartum participants were psychotropic and hormone medication-free English-speaking women aged 18 years or older with a major depressive episode with peripartum onset as defined by SCID-5. The healthy peripartum comparison group (control) included women who had no history of a psychiatric diagnosis and did not develop any psychiatric illness after delivery. 47-52% of the combined sample identified as a racial minority (as defined by NIH). Inclusion criteria for PMD included psychotropic and hormone medication-free English-speaking perimenopausal women defined by the Stages of Reproductive Aging Workshop (STRAW) criteria as being in the early menopause transition (menstrual cycle length varying >7 days in consecutive cycles) or late menopause transition (>60 days without menses but amenorrhea <12 months in the prior 12 months). Inclusion in the study required current mild-to-moderate depressive symptoms, defined as a score from 10 to 25 on the clinician-rated Montgomery-Åsberg Depression Rating Scale (MADRS) and/or a score >7.7 on the self-rated Kellner Symptom Questionnaire Anger-Hostility subscale. The healthy comparison group included perimenopausal women with no psychiatric diagnosis. Inclusion criteria for MDD included men and women, 18 to 65 years of age, who had received a diagnosis of MDD and had a score on the 17-item Hamilton Depression Rating Scale (HAM-D) of 22 or higher (Gunduz-Bruce et al., 2019). Blood samples from well characterized patients with MDD with well documented history of adverse childhood experiences (ACEs) were provided. The healthy comparison group included men and women in this age range without a current psychiatric diagnosis. Exclusion criteria for all groups include individuals with substance use disorders, intellectual disability, severe medical co-morbidity, history of a suicide attempt, history of treatment-resistant depression, and a history of bipolar or psychotic disorder.

Protein Isolation and Quantification from Whole Blood Samples

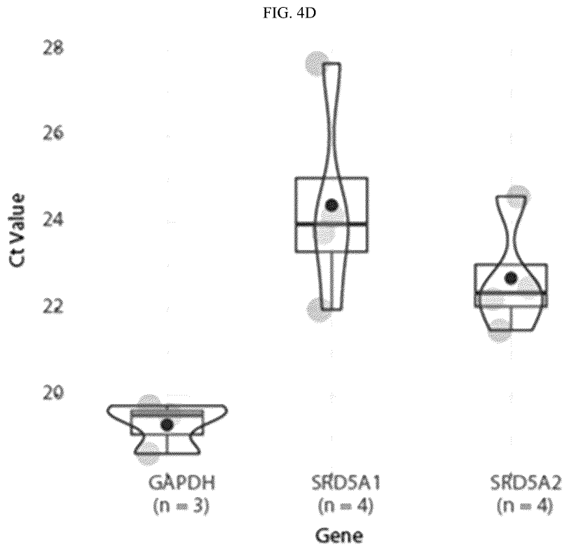

The levels of 5α-reductase were measured in blood samples from patients with depression compared to healthy control subjects. The measurement and quantification of 5α-reductase type 1 and 2 in whole blood samples or serum were performed using both Western blot analysis and qRT-PCR. Briefly, total protein was isolated from whole blood samples by adding an equal volume of RIPA lysis buffer in the presence of phosphatase and protease inhibitors. Protein concentration was determined using a detergent compatible (DC) assay according to manufacturer's instructions and an equal amount of protein was diluted in Laemmli Sample Buffer and loaded onto 12% polyacrylamide gels. Gel electrophoresis was performed, and protein was transferred to an activated polyvinylidene difluoride (PVDF) membrane. Membranes were blocked in 5% milk in TBS-T overnight at 4° C. before being probed with antibodies specific for Srd5a1 (1:1000 Abcam #ab110123), Srd5a2 (1:1000 Abcam #ab27469), or β-actin (1:5000 Sigma A5441) for 2-hours at room temperature, followed by donkey anti-goat HRP (1:2000 Jackson ImmunoResearch #705-035-003). Protein bands were visualized with ECL reagents (ThermoFisher #P132106) and imaged on a ChemiDoc MP (BioRad). Measurements of optical density were calculated using Image J software (NIH) and normalized to β-actin levels, which was previously used as a loading control for plasma-based Western blot analysis. The ability to measure the levels of neurosteroidogenic enzymes in whole blood samples was demonstrated in FIG. 4A.

RNA Extraction and Quantification from Whole Blood Samples

Transcript levels of the genes encoding for 5α-reductase type 1 and type 2, SRD5A1 and SRD5A2, respectively, were measured in whole blood samples. Whole blood samples from subjects were thawed, lysed, and homogenized in guanidine thiocyanate-phenol (TRIzol™) [1000 μL guanidine thiocyanate-phenol per 250 μL of sample]. After allowing vortexed samples to incubate at room temperature (˜25° C.) for 5 minutes, chloroform was added to each sample [200 μL chloroform per 1 mL guanidine thiocyanate-phenol used]. Each sample was vortexed again and incubated at room temperature for 3 minutes. The samples were then centrifuged (12XG, 15 mins at 4° C.) and the RNA in the aqueous phase of each sample was isolated. The RNA was precipitated with isopropanol and glycogen (2 μL) [500 μL isopropanol per 1000 μL guanidine thiocyanate-phenol used]. The samples were mixed well, incubated on wet ice for 10 mins, and centrifuged to pellet (12×G, 30 mins at 4° C.). The RNA pellet was dried and washed in 80% cold ethanol (800 μL), then mixed and centrifuged (7500G, 5 mins at 4° C.). After removal of supernatant, the RNA pellet was dried and resuspended in 1 mM sodium citrate. The samples were heated (15 mins, 60° C.) and cooled before performing quality control.

qRT-PCR was then performed to quantify mRNA levels of Srd5a1 and Srd5a2. In brief, mice were anesthetized with isoflurane, decapitated, the brain rapidly extracted and flash frozen in liquid nitrogen. Tissue punches from the BLA were collected using a 0.5 mm sterile biopsy needle and subsequently homogenized in 25 μL of TRIzol™. Chloroform (15 μl) was then added, samples were centrifuged, and the supernatant was transferred to a sterile tube containing 12.5 μL of cold isopropanol and 2 μL of glycogen. Samples were then pelleted and rinsed in 75% ethanol, air dried, and the resulting RNA product was heated at 60° C. in 1 mM of sodium citrate made up in RNAsecure. RNA integrity and concentration were measured using a ThermoScientific NanoDrop™ 1000. SuperScript™ III Platinum™ SYBR™ Green (one step qRT PCR kit) (Invitrogen Lot No. 2028735) was used for SYBR-based qRT-PCR along with 100 ng of template RNA and primers listed in TABLE 1. Only samples with acceptable RNA integrity (≥1.8 260:280 ratio) will be used for SYBR-based qRT-PCR along with 100 ng of template RNA and primers. A Stratagene Mx3000P™ (Agilent Genomics, Santa Clara, CA, USA) was used to perform the qRT-PCR using the following thermocycling parameters: 1 cycle at 50° C. for 3 minutes, 1 cycle at 95° C. for 5 minutes, 40 cycles of 15 seconds at 95° C. followed by 40 seconds at 60° C., 1 cycle at 40° C. for 1 minute, 1 cycle at 95° C. for 1 minute followed by 30 seconds at 55° C. and 30 seconds at 95° C. All samples were run in duplicate with an average CT value normalized to GAPDH. Relative transcript levels were calculated in accordance with the 2-ΔΔCT method. See, FIGS. 4B-4D.

TABLE 1

| NAME | PRIMER SEQUENCE |
|---|---|
| SRD5A1: | |
| Forward | CAGACCCTGTCTGCTGTTATC (SEQ ID NO: 1) |
| Reverse | GATAACAGCAGACAGGGTCTG (SEQ ID NO: 2) |
| Forward | GAGATATTCAGCTGAGACCC (SEQ ID NO: 3) |
| Reverse | TTAGTATGTGGGCAGCTTGG (SEQ ID NO: 4) |
| SRD5A2: | |
| Forward | CCCTTTGAGGGTCAGATGTATG (SEQ ID NO: 5) |
| Reverse | CATACATCTGACCCTCAAAGGG (SEQ ID NO: 6) |
| Forward | ATTTGTGTGGCAGAGAGAGG (SEQ ID NO: 7) |
| Reverse | TTGATTGACTGCCTGGATGG (SEQ ID NO: 8) |
| TSPO: | |
| Forward | TCCTACCTGGTCTGGAAAGAG (SEQ ID NO: 9) |
| Reverse | CTCTTTCCAGACCAGGTAGGA (SEQ ID NO: 10) |
| CYP11A1: | |
| Forward | CCAGAAGTATGGCCCGATTTA (SEQ ID NO: 11) |
| Reverse | TAAATCGGGCCATACTTCTGG (SEQ ID NO: 12) |
| AKR1C1: | |
| Forward | GGGCAGGTGACAGGTATTTAT (SEQ ID NO: 13) |
| Reverse | ATAAATACCTGTCACCTGCCC (SEQ ID NO: 14) |
| AKR1C2: | |
| Forward | CGGGTTCCACCATATTGATTCT (SEQ ID NO: 15) |
| Reverse | AGAATCAATATGGTGGAACCCG (SEQ ID NO: 16) |
| AKR1C3: | |
| Forward | CCAGAGGTTCCGAGAAGTAAAG (SEQ ID NO: 17) |
| Reverse | CTTTACTTCTCGGAACCTCTGG (SEQ ID NO: 18) |
| AKR1C4: | |
| Forward | GGAGGTCATGGAGAAGTGTAAG (SEQ ID NO: 19) |
| Reverse | CTTACACTTCTCCATGACCTCC (SEQ ID NO: 20) |
| HSD3B2: | |
| Forward | ACTCAGCCCAATTTACTCCTATC (SEQ ID NO: 21) |
| Reverse | GATAGGAGTAAATTGGGCTGAGT (SEQ ID NO: 22) |
| GAPDH: | |
| Forward | CGGAGTCAACGGATTTGGTCGTAT (SEQ ID NO: 23) |
| Reverse | ATACGACCAAATCCGTTGACTCCG (SEQ ID NO: 24) |

### Example 5: Relationship Between 5α-Reductase Expression and Endogenous Neurosteroidogenesis

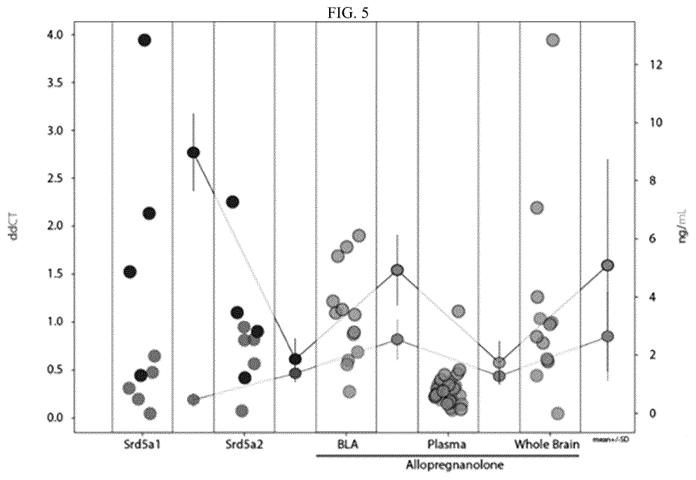

Srd5a1 and Srd5a2 expression was decreased in mice subjected to chronic unpredictable stress (CUS) (FIG. 5: ddCT: ˜ 0-1; 0-3 ng/ml; cols. 1-2, from left to right) compared to control (FIG. 5: ddCT: ˜0.5-4; ˜ 2-13 ng/ml; cols. 1-2, from left to right). The reductions in Srd5a1 following CUS were larger than the changes in Srd5a2.

The decrease in Srd5a1 and Srd5a2 expression following CUS was correlated with a decrease in allopregnanolone levels in the BLA (FIG. 5: ddCT: ˜ 0.25-0.75; 1-2 ng/ml; col. 3) and brain (FIG. 5: ddCT: ˜ 0-1; 0-3 ng/ml; col. 5), but not plasma (FIG. 5: ddCT: ˜ 0-0.5; 0-1.5 ng/ml; col. 4) compared to control without CUS (BLA (FIG. 5: ddCT: ˜ 0.75-2; 3-6 ng/ml; col. 3); brain (FIG. 5: ddCT: ˜ 0.5-4; 2-13 ng/ml; col. 5); plasma (FIG. 5: ddCT: ˜ 0-1; 0-4 ng/ml; col. 4)). The mean±standard deviation for each transcript expression was calculated and shown in FIG. 5 after each column. The mean dot following CUS is represented by the lower dot, while the upper dot represented control without CUS, while standard deviation is shown by the error bars. These data demonstrate the relationship between the expression of Srd5a1 and Srd5a2 and the capacity for endogenous neurosteroidogenesis. These data demonstrated the limitation of measuring allopregnanolone levels in the blood as a measure for the capacity for endogenous neurosteroidogenesis in the brain. Thus, measuring the expression of the enzymes involved in neurosteroidogenesis, such as 5α-reductase, provided an improvement over conventional approaches for characterizing neurosteroidogenesis.

### Example 6: Subpopulation Identification of Individuals with Reduced Endogenous Neurosteroidogenesis

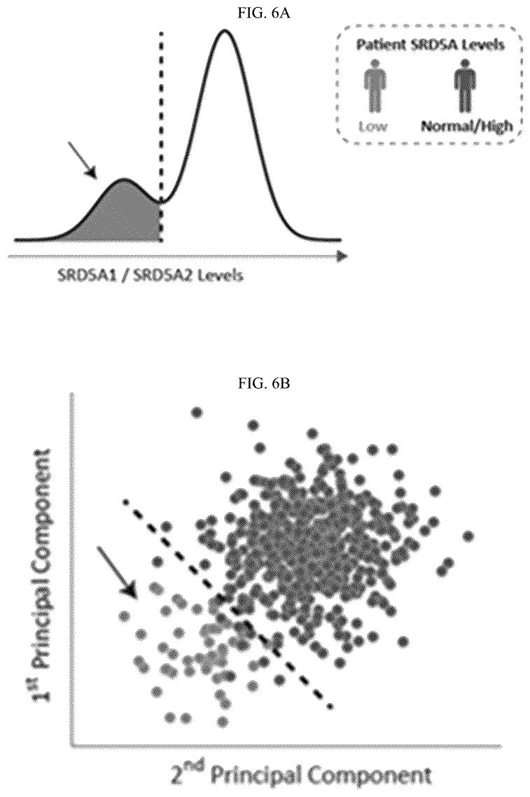

A computational approach may be used to stratify patients with a reduced capacity for endogenous neurosteroidogenesis. A reduction of 5α-reductase expression using Western blot and qRT-PCR for both 5α-reductase 1 and 2 (SRD5A1 and SRD5A2) as quantified in FIG. 4, indicated a reduced capacity for endogenous neurosteroid synthesis. In order to detect the subset or subpopulation of patients having a reduced capacity of endogenous neurosteroidogenesis, the distributions of each parameter are examined. The distribution of the patient samples are analyzed to identify patients with a reduced capacity for endogenous neurosteroidogenesis. This approach is expected to identify two distinct populations, one with lower average levels of 5α-reductase expression (FIG. 6A, left peak) versus normal or high levels of 5α-reductase (FIG. 6A, right peak) (bimodal (Normal: Peak Separation). In another approach, clusters in the data are detected by performing dimensionality reduction using PCA and using unsupervised clustering methods, such as Gaussian mixture model (GMM) and density-based spatial clustering of applications with noise (DBSCAN) (FIG. 6B). Lower average levels of 5α-reductase expression would be found in the lower left corner of the graph, i.e., below or to the left of the dashed line separating the lower average levels from the normal or high levels of 5α-reductase expression found to the right of the dashed line. In another approach, a distribution metric (such as z-scores or percentiles) is used to identify patients that lie at the lower end of the distribution (FIG. 6C). The unimodal (normal: Z-score) peak shows lower average levels of 5α-reductase expression to the left of the dashed line and to the area the arrow points (FIG. 6C, Left side). The unimodal (skewed: percentile) peak shows lower average levels of 5α-reductase expression to the left of the dashed line and to the area the arrow points (FIG. 6C, Right side). A subset of patients with PPD, PMD, and MDD is expected to exhibit reduced expression of 5α-reductase type 1 or 2 at the protein level and/or transcript level. Accordingly, any changes in either 5α-reductase type 1 or 2 reflects reduced capacity for endogenous neurosteroidogenesis. There may be sex differences in the baseline expression of 5α-reductase type 1 or 2 between males and females. Females may be more susceptible to a reduced capacity for endogenous NAS synthesis, such as in conditions of altered ovarian hormone and NAS derivatives during the peripartum or perimenopausal periods. Using these methods, subsets of patients with a reduced capacity for endogenous neurosteroid synthesis would be identified and responsive to NAS-based treatments.

### Example 7: Relationship Between 5α-Reductase Expression and Previous Trauma Exposure

Preclinical data demonstrated a reduced capacity for endogenous neurosteroidogenesis due to a reduction in the expression of 5α-reductase type 1 and 2 following chronic stress exposure, a major risk factor for depression. To determine whether previous trauma exposure may contribute to a reduced capacity for endogenous neurosteroidogenesis in a subset of patients with depression, the relationship between the extent of adverse childhood experiences (ACEs) which are documented in the well-characterized patient populations and the expression of 5α-reductase type 1 and 2 is examined. The relationship between 5α-reductase expression and proximal stress exposure documented in the patient history is also examined. Samples from patients diagnosed with PPD, PMD, MDD, and individuals without a psychiatric diagnosis (controls) will be used to measure protein and transcript levels of 5α-reductase type 1 and 2 and the levels will be correlated with the number of ACEs and/or proximal stressors in individuals with PPD, PMD, or MDD. Individuals with a higher number of ACEs (≥4), about 25% of the population, will have lower levels of 5α-reductase and individuals with a lower number of ACEs (≤3) and/or proximal stress exposure will have higher 5α-reductase levels.

Levels of 5α-reductase associated with vulnerability to depression is also tested. The expression of 5α-reductase across individuals with an equal number of ACEs and/or proximal stress exposure that developed depression (high risk) compared to those that did not (low risk) are examined. Protein and transcript levels of 5α-reductase 1 and 2 will be measured in patients with a diagnosis of depression and individuals with no psychiatric diagnosis with documented levels of ACEs. Individuals with a high number of ACEs and a diagnosis of depression (high risk) exhibit lower levels of 5α-reductase. In contrast, individuals with a high number of ACEs, but no diagnosis of depression (low risk) will exhibit higher levels of 5α-reductase expression.

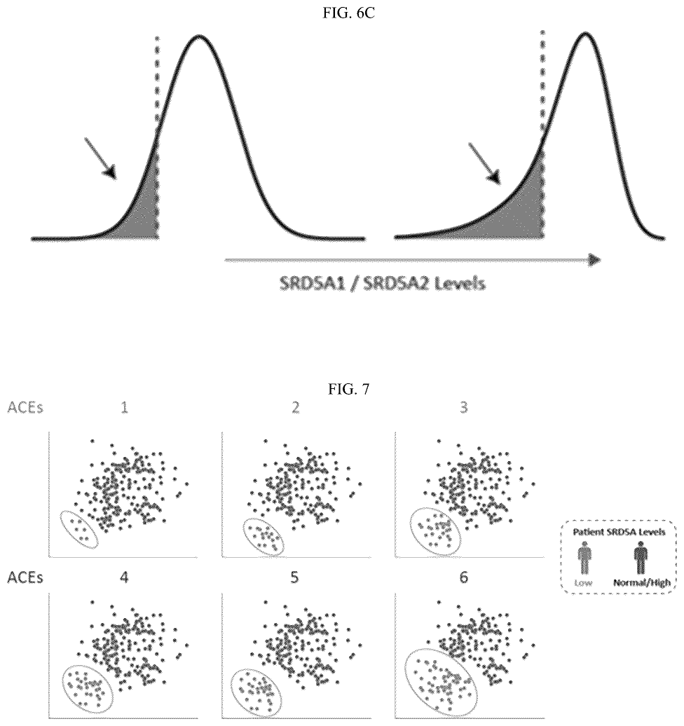

Computational approaches are used to analyze the distribution of 5α-reductase. Clusters identified by performing dimensionality reduction using PCA and using unsupervised clustering methods such as GMM and DBSCAN for 5α-reductase levels in relation to the number of ACEs will reveal distinct populations in which a subset of individuals with a high number of ACEs and low 5α-reductase expression will be distinct from individuals which cluster with low ACEs and high 5α-reductase expression (FIG. 7). In addition, Individuals with a high number of ACEs and a depression diagnosis will have low levels of 5α-reductase; whereas, individuals with a high number of ACEs but no depression diagnosis will retain a high level of 5α-reductase expression (FIG. 7). A similar approach will be used for proximal stress exposure.

Generalized estimating equation (GEE) models will be used to examine the association of depressive symptoms with the levels of 5α-reductase type 1 and 2. A similar approach will be used to determine the association between the number of ACEs/proximal stress and the expression of 5α-reductase type 1 and 2. The GEE models are used to determine the association of depression symptoms (HAM-D, MADRAS) with each of the outcome measures (protein and transcript levels of 5α-reductase type 1 and 2) and then each of the following a priori covariates: age, race, body mass index (BMI), marital status, education, lifetime history of depression, and number of stressful life events. Multivariate models will be developed by adjusting first for the other primary predictors and covariates examined that achieve the same significance threshold.

### Example 8: Effectiveness of Biomarker Predicting Treatment Response to NAS-Based Treatments

To determine whether patient stratification achieved predicts treatment response to NAS-based treatments and whether a reduced capacity for endogenous neurosteroidogenesis predicts treatment response to NAS-based treatments, participants are recruited using the following methods: word of mouth, referrals, advertisements, and current patient enrollment. Inclusion criteria for PPD includes English-speaking women age 18 years or older with a SCID diagnosis of a major depressive episode with peripartum onset, with a score on the 17-item Hamilton Depression Rating Scale (HAM-D) of 22 or higher and are within 6 months of delivery. Inclusion criteria for PMD includes English-speaking perimenopausal women defined by the Stages of Reproductive Aging Workshop (STRAW) criteria as being in the early menopause transition (menstrual cycle length varying >7 days in consecutive cycles) or late menopause transition (>60 days without menses but amenorrhea <12 months in the prior 12 months). Inclusion in the PMD study requires current mild-to-moderate depressive symptoms, defined as a score on the 17-item Hamilton Depression Rating Scale (HAM-D) of 22 or higher. Similarly, inclusion criteria for MDD will include both men and women, 18 to 65 years of age, who have received a diagnosis of major depressive disorder with a score on the 17-item Hamilton Depression Rating Scale (HAM-D) of 22 or higher (Gunduz-Bruce et al., 2019). Exclusion criteria for all groups include individuals with current psychotic symptoms, current psychotropic medications, pregnancy or lactation, substance use disorders, intellectual disability, severe medical co-morbidity, history of a recent suicide attempt, treatment-resistant depression, bipolar disorder, or any psychotic disorder. The number of patients enrolled in the study will be based on the distribution of patients. However, based on the evidence that 57% of women with PPD exhibit a rapid antidepressant response with zuranolone treatment, which is associated with a reduced capacity for endogenous neurosteroidogenesis, a relatively equal split between responders and non-responders is anticipated. Based on these assumptions, a power analysis suggests that 50 depressed patients in each clinical context should be sufficient for the study.

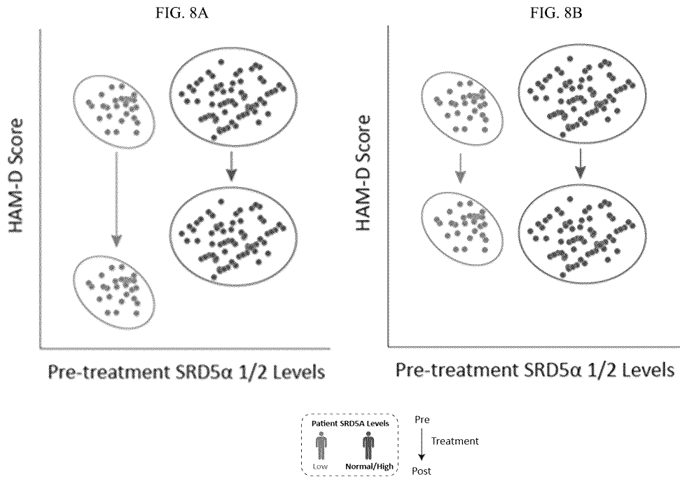

Blood samples are drawn at the initiation of the study, at the conclusion of treatment (15 day), and at the conclusion of the study (30 days) following treatment with zuranolone, a NAS-based treatment. Zuranolone (50 mg dose) is administered at 8 p.m. with food on days 1 through 14. Patients who report moderate sedation as defined in the clinical trials, will have the dose reduced to 40 mg. The primary endpoint is the change from baseline (CFB) in HAM-D score at day 15. The secondary endpoints include the CFB in the score on Bech-6 (a subscale of HAM-D that assesses the overall severity of depression on the basis of ratings in six categories [depressed mood, feelings of guilt, work and activities, psychomotor retardation, psychic anxiety, and general somatic symptoms], the CFB in the Hamilton Anxiety Rating Scale total score (HAM-A; scores range from 0 to 56, with higher scores indicating more severe anxiety) and CFB in HAM-D total score at the conclusion of treatment (day 15). Patients are grouped based on treatment response as “Responders” (≥50% reduction in HAM-D) or “Non-Responders” to zuranolone. The expression of 5α-reductase at the initiation of the study is compared between “Responders” and “Non-Responders” to zuranolone. Further, the relationship between 5α-reductase expression in those patients who achieve remission and in relationship to previous trauma exposure (ACEs) and/or proximal stress exposure are examined. Individuals with low expression of 5α-reductase at the initiation of the study will exhibit a greater treatment response to zuranolone (“Responders”) than those with higher levels of 5α-reductase (“Non-Responders”) (FIGS. 8A-8B). There may be sex differences in baseline expression of 5α-reductase expression in males and females which may influence their responsiveness to treatment. Although it is not a primary endpoint of the current proposal, the relationship between 5α-reductase expression at the conclusion of the trials and the treatment response to zuranolone will be examined to explore the utility of this approach as a potential response biomarker, given the persistent antidepressant effects of these compounds, lasting up to 45 days after treatment.

The least-squares mean CFB in HAM-D, Bech-6, and HAM-A total scores are measured and the GEE models are used to determine the association with the outcome measures of interest (protein and transcript levels of 5α-reductase type 1 and 2) and then each of the following a priori covariates: sex, age, race, body mass index (BMI), marital status, education, lifetime history of depression, and number of stressful life events (ACEs and proximal stressors). For each of the univariate models for which the predictor variable was statistically significant, multivariate models will be developed by adjusting first for the other primary predictors.

### Example 9: qRT-PCR Analysis

SRD5A1 and SRD5A2 RNA levels were quantified using the qRT-PCR method. The qRT-PCR quantified the cycles (Ct) required to reach a threshold at which the fluorescence signal indicated exponential amplification of DNA. Ct values inversely correlate with gene expression levels, with lower Ct values corresponding to higher expression levels and vice versa. Within each plate, the Ct for each SRD5A gene was normalized to the housekeeping (GAPDH) gene (SRD5A-GAPDH) to obtain ΔCt values, accounting for technical variability. ACt values thus are inversely proportional to gene expression accounting for technical variability. Two combined measures of SRD5A expression were added: 1) Total SRD5A ΔCt (SRD5A1 ΔCt+SRD5A2 ΔCt) and 2) SRD5A ratio (SRD5A1 ΔCt+SRD5A2 ΔCt). Within each plate each sample was measured in duplicate to obtain a measure of intra-plate biological variability. Additionally, one pair of identical samples from each plate was also quantified on another plate to measure inter-plate biological variability. Thus, intra-plate to inter-plate biological variability was compared and no significant differences were found. To examine whether differences in SRD5A expression levels are associated with altered behavioral outcomes [STATE, TRAIT, CAPs], the expression of SRD5A in the blood samples were divided into groups: Low Expression (ΔCt values above the 90th percentile) and the general population. These analyses were conducted separately for 1) groups stratified by sex at birth, and 2) for combined sexes.

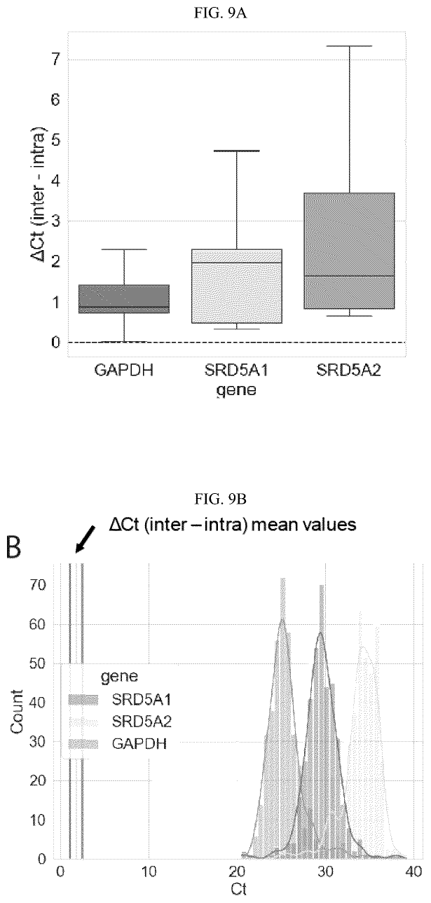

Analyses using qRT-PCR were performed to demonstrate 1) the validation of the biomarker assay; 2) the ability to stratify patients based on SRD5A expression; 3) the relationship with anxiety symptoms; and 4) the relationship with PTSD symptoms. See, e.g., FIGS. 9A-9B; 10A-10B, 11A-11B, and 12A-12B.

### Other Embodiments

From the foregoing description, it will be apparent that variations and modifications may be made to the disclosure described herein to adopt it to various usages and conditions. Such embodiments are also within the scope of the following claims.

Embodiment 1. A panel of polypeptide or polynucleotide biomarkers, wherein the biomarkers are selected from the group consisting of: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKRIC2); aldo-keto reductase family 1, member C3 (AKRIC3); aldo-keto reductase family 1, member C4 (AKRIC4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

Embodiment 2. The panel of embodiment 1, wherein the biomarkers are bound by a capture molecule.

Embodiment 3. The panel of embodiment 2, wherein the capture molecule is bound to a substrate.

Embodiment 4. The panel of embodiment 3, wherein the capture molecule is a polypeptide or polynucleotide.

Embodiment 5. The panel of embodiment 3, wherein the polypeptide is an antibody or antigen binding fragment thereof.

Embodiment 6. A method of treating a subject selected as having a reduced capacity for neurosteroidogenesis, the method comprising administering to the selected subject either exogenous neurosteroids or an agent that increases endogenous neurosteroidogenesis, wherein the subject is selected by detecting a reduction in a biomarker in a sample of the subject relative to a reference, wherein the biomarker selected from the group consisting of steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKRIC4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

Embodiment 7. A method of characterizing a subject having or having a propensity to develop a psychiatric illness, the method comprising measuring levels of a biomarker in a sample from the subject compared to a reference, wherein the biomarker is selected from the group consisting of steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKRIC2); aldo-keto reductase family 1, member C3 (AKR1C3); aldo-keto reductase family 1, member C4 (AKRIC4); and hydroxy-delta-5-steroid dehydrogenase, 3 beta- and steroid delta-isomerase 2 (HSD3B2), thereby characterizing the subject as having or having a propensity to develop a psychiatric illness.

Embodiment 8. The method of embodiment 7, wherein detection of a reduction in the level of the marker is indicative that the subject has or has a propensity to develop a psychiatric illness.

Embodiment 9. The method of embodiment 7, wherein failure to detect a reduction in the level of the marker is indicative that the subject does not have or does not have a propensity to develop a psychiatric illness.

Embodiment 10. A method of determining the efficacy of a medicament or monitoring the efficacy of a medicament for treating a psychiatric illness in a subject, the method comprising a) measuring levels of a biomarker in a sample from the subject prior to administration of the medicament, wherein the biomarker is selected from the group consisting of steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKRIC2); aldo-keto reductase family 1, member C3 (AKR1C3); aldo-keto reductase family 1, member C4 (AKR1C4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR);
b) measuring levels of the biomarkers in a second sample from the subject following treatment with the medicament, wherein an increase in the level of the biomarker in the first sample relative to the second sample indicates that the medicament is efficacious for treating the psychiatric illness.

Embodiment 11. The method of any one of embodiments 6-10, wherein the measuring comprises
a) isolating total protein from the sample from the subject and from a reference sample;
b) detecting antibody binding to a biomarker present in the sample of the subject relative to antibody binding to the reference sample; and
c) quantifying protein expression.

Embodiment 12. The method of any one of embodiments 6-10, wherein the measuring comprises:
isolating RNA from the sample from the subject and from the sample from the reference;
adding biomarker-specific primers and test control-specific primers (e.g., β-actin; GAPDH); and
quantifying levels of expression.

Embodiment 13. The method of any one of embodiments 6 and 10-12, wherein the treating comprises administering a medicament selected from the group consisting of steroid 5 alpha-reductase 1 (SRD5A1); steroid 5 alpha-reductase 2 (SRD5A2); translocator protein (TSPO); cytochrome P450 family 11 subfamily A member 1 (CYP11A1); aldo-keto reductase 1C superfamily (e.g., ALRIC1, AKRIC2, AKRIC3, AKRIC4); 3β-hydroxysteroid dehydrogenase (3-β-HSD (e.g., hydroxy-delta-5-steroid dehydrogenase, 3-beta- and steroid deltaisomerase 2 (HSD3B2)); Steroidogenic acute regulatory protein (StAR); positive allosteric modulator of GABAA receptors (e.g., ganaxolone (ZTALMY®); brexanolone (ZULRESSOR); zuranolone (ZURZUVAE™)); progesterone analogue (e.g., (8S,9S,10R,13S,14S,17S)-17-((E)-1-(Hydroxyimino)ethyl)-10,13-dimethyl-6,7,8,9,10,11,12,13,14,15,16,17-dodecahydro-1H-cyclopenta[a]phenanthren-3 (2H)-one (EIDD-036)); allopregnanolone prodrug (e.g., LYT-300); brexanolone prodrug (e.g., ester prodrugs, carbonate prodrugs); and delta-subunit-selective GABAA receptor modulator (e.g., DS2).

Embodiment 14. The method of any one of embodiments 6-13, wherein the psychiatric illness is selected from the group consisting of depression; anxiety disorders; post-traumatic stress disorder (PTSD); premenstrual dysphoric disorder (PMDD); traumatic brain injury; concussion; and epilepsy.

Embodiment 15. The method of embodiment 14, wherein the psychiatric illness is selected from the group consisting of postpartum depression, peripartum depression, perimenopausal depression, major depression; infertility-related depression; generalized anxiety disorder; panic disorder; social anxiety disorder; specific or simple phobias; agoraphobia; separation anxiety disorder; selective mutism; and catamenial epilepsy.

Embodiment 16. The method of embodiment 12, wherein the biomarker-specific primers include primer sets selected from the group consisting of:

SRD5A1:
Forward
(SEQ ID NO: 1)
CAGACCCTGTCTGCTGTTATC
Reverse
(SEQ ID NO: 2)
GATAACAGCAGACAGGGTCTG
Forward
(SEQ ID NO: 3)
GAGATATTCAGCTGAGACCC
Reverse
(SEQ ID NO: 4)
TTAGTATGTGGGCAGCTTGG
SRD5A2:
Forward
(SEQ ID NO: 5)
CCCTTTGAGGGTCAGATGTATG
Reverse
(SEQ ID NO: 6)
CATACATCTGACCCTCAAAGGG
Forward
(SEQ ID NO: 7)
ATTTGTGTGGCAGAGAGAGG
Reverse
(SEQ ID NO: 8)
TTGATTGACTGCCTGGATGG
TSPO:
Forward
(SEQ ID NO: 9)
TCCTACCTGGTCTGGAAAGAG
Reverse
(SEQ ID NO: 10)
CTCTTTCCAGACCAGGTAGGA
CYP11A1:
Forward
(SEQ ID NO: 11)
CCAGAAGTATGGCCCGATTTA
Reverse
(SEQ ID NO: 12)
TAAATCGGGCCATACTTCTGG
AKR1C1:
Forward
(SEQ ID NO: 13)
GGGCAGGTGACAGGTATTTAT
Reverse
(SEQ ID NO: 14)
ATAAATACCTGTCACCTGCCC
AKR1C2:
Forward
(SEQ ID NO: 15)
CGGGTTCCACCATATTGATTCT
Reverse
(SEQ ID NO: 16)
AGAATCAATATGGTGGAACCCG
AKR1C3:
Forward
(SEQ ID NO: 17)
CCAGAGGTTCCGAGAAGTAAAG
Reverse
(SEQ ID NO: 18)
CTTTACTTCTCGGAACCTCTGG
AKR1C4:
Forward
(SEQ ID NO: 19)
GGAGGTCATGGAGAAGTGTAAG
Reverse
(SEQ ID NO: 20)
CTTACACTTCTCCATGACCTCC
HSD3B2:
Forward
(SEQ ID NO: 21)
ACTCAGCCCAATTTACTCCTATC
Reverse
(SEQ ID NO: 22)
GATAGGAGTAAATTGGGCTGAGT.

Embodiment 17. The method of embodiment 12, wherein the test control-specific primers are:

GAPDH:
Forward
(SEQ ID NO: 23)
CGG AGT CAA CGG ATT TGG TCG TAT
Reverse
(SEQ ID NO: 24)
ATA CGA CCA AAT CCG TTG ACT CCG.

Embodiment 18. The method of any one of embodiments 6-17, wherein the sample is selected from the group consisting of: blood, serum, urine, and a buccal smear.

Embodiment 19. The method of any one of embodiments 6-18, wherein the reference is a housekeeping protein or a housekeeping gene.

Embodiment 20. The method of embodiment 19, wherein the housekeeping protein is β-actin or Glyceraldehyde-3-phosphate dehydrogenase (GAPDH).

Embodiment 21. A kit comprising a capture molecule that specifically binds a biomarker selected from the group consisting of steroid 5 alpha-reductase 1 (SRD5A1); steroid 5 alpha reductase 2 (SRD5A2); translocator protein (TSPO); Glyceraldehyde 3-phosphate dehydrogenase (GAPDH); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKR1C4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

Embodiment 22. The kit of embodiment 21, wherein the capture molecule is a polynucleotide or polypeptide.

Embodiment 23. The kit of embodiment 21, wherein the kit comprises a primer set and a detectably labeled probe that specifically amplifies and detects at least one or more biomarkers selected from the group consisting of steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKRIC4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

Embodiment 24. The kit of embodiment 22, wherein the capture molecule is an antibody that specifically binds the biomarker.

Embodiment 25. The kit of embodiment 21, further comprising reverse transcriptase and/or thermostable DNA polymerase.

Embodiment 26. The kit of embodiment 21, further comprising an enzyme with both reverse transcriptase and DNA polymerase activity.

Embodiment 27. A method of treating a subject having a psychiatric illness characterized by reduced levels of one or more biomarkers of endogenous neurosteroidogenesis, comprising:
(a) detecting expression of the one or more biomarkers in a sample obtained from the subject by contacting the sample with an antibody against the one or more biomarkers and determining if the antibody bound to the one or more biomarkers;
(b) comparing expression of the one or more biomarkers in the sample to a reference from a healthy control subject; and
(c) administering a neuroactive steroid-based agent to the subject if the sample has reduced levels of one or more biomarkers of endogenous neurosteroidogenesis as compared to the reference.

Embodiment 28. The method of embodiment 27, wherein the one or more biomarkers is selected from the group consisting of steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKRIC3); aldo-keto reductase family 1, member C4 (AKRIC4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR).

Embodiment 29. The method of embodiment 27 or embodiment 28, wherein the neuroactive steroid-based agent is selected from the group consisting of steroid 5 alpha-reductase 1 (SRD5A1); steroid 5 alpha-reductase 2 (SRD5A2); translocator protein (TSPO); cytochrome P450 family 11 subfamily A member 1 (CYP11A1); aldo-keto reductase 1C superfamily (e.g., ALRIC1, AKRIC2, AKR1C3, AKRIC4); 3β-hydroxysteroid dehydrogenase (3-β-HSD (e.g., hydroxy-delta-5-steroid dehydrogenase, 3-beta- and steroid deltaisomerase 2 (HSD3B2)); Steroidogenic acute regulatory protein (StAR); positive allosteric modulator of GABAA receptors (e.g., ganaxolone (ZTALMY®); brexanolone (ZULRESSOR); zuranolone (ZURZUVAE™)); progesterone analogue (e.g., (8S,9S,10R,13S,14S,17S)-17-((E)-1-(Hydroxyimino)ethyl)-10,13-dimethyl-6,7,8,9,10,11,12,13,14,15,16,17-dodecahydro-1H-cyclopenta[a]phenanthren-3 (2H)-one (EIDD-036)); allopregnanolone prodrug (e.g., LYT-300); brexanolone prodrug (e.g., ester prodrugs, carbonate prodrugs); and delta-subunit-selective GABAA receptor modulator (e.g., DS2).

Embodiment 30. The method of any one of embodiments 27-29, wherein the psychiatric illness is selected from the group consisting of depression; anxiety disorders; post-traumatic stress disorder (PTSD); premenstrual dysphoric disorder (PMDD); traumatic brain injury; concussion; and epilepsy.

Embodiment 31. The method of embodiment 30, wherein the psychiatric illness is selected from the group consisting of postpartum depression, peripartum depression, perimenopausal depression, major depression; infertility-related depression; generalized anxiety disorder; panic disorder; social anxiety disorder; specific or simple phobias; agoraphobia; separation anxiety disorder; selective mutism; and catamenial epilepsy.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A panel of polypeptide or polynucleotide biomarkers, wherein the biomarkers are selected from the group consisting of: steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKR1C3); aldo-keto reductase family 1, member C4 (AKR1C4); 3β-hydroxysteroid dehydrogenase (3-β-HSD); and Steroidogenic acute regulatory protein (StAR).

2. The panel of claim 1, wherein the biomarkers are bound by a capture molecule.

3. The panel of claim 2, wherein the capture molecule is bound to a substrate.

4. The panel of claim 3, wherein the capture molecule is a polypeptide or polynucleotide.

5. The panel of claim 3, wherein the polypeptide is an antibody or antigen binding fragment thereof.

6. A method of characterizing a subject having or having a propensity to develop a psychiatric illness, the method comprising measuring levels of a biomarker in a sample from the subject compared to a reference, wherein the biomarker is selected from the group consisting of steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKR1C1); aldo-keto reductase family 1, member C2 (AKR1C2); aldo-keto reductase family 1, member C3 (AKR1C3); aldo-keto reductase family 1, member C4 (AKR1C4); and hydroxy-delta-5-steroid dehydrogenase, 3 beta- and steroid delta-isomerase 2 (HSD3B2), thereby characterizing the subject as having or having a propensity to develop a psychiatric illness.

7. The method of claim 6, wherein detection of a reduction in the level of the marker is indicative that the subject has or has a propensity to develop a psychiatric illness.

8. The method of claim 6, wherein failure to detect a reduction in the level of the marker is indicative that the subject does not have or does not have a propensity to develop a psychiatric illness.

9. The method of claim 6, wherein the measuring comprises:
a) isolating total protein from the sample from the subject and from a reference sample;
b) detecting antibody binding to a biomarker present in the sample of the subject relative to antibody binding to the reference sample; and
c) quantifying protein expression.

10. The method of claim 6, wherein the measuring comprises:
isolating RNA from the sample from the subject and from the sample from the reference;
adding biomarker-specific primers and test control-specific primers; and
quantifying levels of expression.

11. The method of claim 6, wherein the method further comprises administering to the subject having or having a propensity to develop a psychiatric illness a therapeutically effective amount of a medicament selected from the group consisting of steroid 5 alpha-reductase 1 (SRD5A1); steroid 5 alpha-reductase 2 (SRD5A2); translocator protein (TSPO); cytochrome P450 family 11 subfamily A member 1 (CYP11A1); aldo-keto reductase 1C superfamily; 3β-hydroxysteroid dehydrogenase (3-β-HSD); Steroidogenic acute regulatory protein (StAR); positive allosteric modulator of GABAA receptors; progesterone analogue; allopregnanolone prodrug; brexanolone prodrug; and delta-subunit-selective GABAA receptor modulator, thereby treating the subject.

12. The method of claim 6, wherein the psychiatric illness is selected from the group consisting of depression; anxiety disorders; post-traumatic stress disorder (PTSD); premenstrual dysphoric disorder (PMDD); traumatic brain injury; concussion; and epilepsy.

13. The method of claim 12, wherein the psychiatric illness is selected from the group consisting of postpartum depression, peripartum depression, perimenopausal depression, major depression; infertility-related depression; generalized anxiety disorder; panic disorder; social anxiety disorder; specific or simple phobias; agoraphobia; separation anxiety disorder; selective mutism; and catamenial epilepsy.

14. The method of claim 10, wherein the biomarker-specific primers include primer sets selected from the group consisting of:
SRD5A1:
Forward
(SEQ ID NO: 1)
CAGACCCTGTCTGCTGTTATC
Reverse
(SEQ ID NO: 2)
GATAACAGCAGACAGGGTCTG
Forward
(SEQ ID NO: 3)
GAGATATTCAGCTGAGACCC
Reverse
(SEQ ID NO: 4)
TTAGTATGTGGGCAGCTTGG
SRD5A2:
Forward
(SEQ ID NO: 5)
CCCTTTGAGGGTCAGATGTATG
Reverse
(SEQ ID NO: 6)
CATACATCTGACCCTCAAAGGG
Forward
(SEQ ID NO: 7)
ATTTGTGTGGCAGAGAGAGG
Reverse
(SEQ ID NO: 8)
TTGATTGACTGCCTGGATGG
TSPO:
Forward
(SEQ ID NO: 9)
TCCTACCTGGTCTGGAAAGAG
Reverse
(SEQ ID NO: 10)
CTCTTTCCAGACCAGGTAGGA
CYP11A1:
Forward
(SEQ ID NO: 11)
CCAGAAGTATGGCCCGATTTA
Reverse
(SEQ ID NO: 12)
TAAATCGGGCCATACTTCTGG
AKR1C1:
Forward
(SEQ ID NO: 13)
GGGCAGGTGACAGGTATTTAT
Reverse
(SEQ ID NO: 14)
ATAAATACCTGTCACCTGCCC
AKR1C2:
Forward
(SEQ ID NO: 15)
CGGGTTCCACCATATTGATTCT
Reverse
(SEQ ID NO: 16)
AGAATCAATATGGTGGAACCCG
AKR1C3:
Forward
(SEQ ID NO: 17)
CCAGAGGTTCCGAGAAGTAAAG
Reverse
(SEQ ID NO: 18)
CTTTACTTCTCGGAACCTCTGG
AKR1C4:
Forward
(SEQ ID NO: 19)
GGAGGTCATGGAGAAGTGTAAG
Reverse
(SEQ ID NO: 20)
CTTACACTTCTCCATGACCTCC
HSD3B2:
Forward
(SEQ ID NO: 21)
ACTCAGCCCAATTTACTCCTATC
Reverse
(SEQ ID NO: 22)
GATAGGAGTAAATTGGGCTGAGT.

15. The method of claim 10, wherein the test control-specific primers are:
GAPDH:
Forward
(SEQ ID NO: 23)
CGG AGT CAA CGG ATT TGG TCG TAT
Reverse
(SEQ ID NO: 24)
ATA CGA CCA AAT CCG TTG ACT CCG

16. The method of claim 6, wherein the sample is selected from the group consisting of: blood, serum, urine, and a buccal smear.

17. The method of claim 6, wherein the reference is a housekeeping protein or a housekeeping gene.

18. The method of claim 17, wherein the housekeeping protein is β-actin or Glyceraldehyde-3-phosphate dehydrogenase (GAPDH).

19. A method of treating a subject having a psychiatric illness characterized by reduced levels of one or more biomarkers of endogenous neurosteroidogenesis, comprising:
(a) detecting expression of the one or more biomarkers in a sample obtained from the subject by contacting the sample with an antibody against the one or more biomarkers and determining if the antibody bound to the one or more biomarkers;
(b) comparing expression of the one or more biomarkers in the sample to a reference from a healthy control subject; and
(c) administering a neuroactive steroid-based agent to the subject if the sample has reduced levels of one or more biomarkers of endogenous neurosteroidogenesis as compared to the reference, thereby treating the subject.

20. The method of claim 19, wherein the one or more biomarkers is selected from the group consisting of steroid 5 alpha-reductase 1 (SRD5A1); steroid 5 alpha reductase 2 (SRD5A2); translocator protein (TSPO); cytochrome P450 family 11, subfamily A, polypeptide 1 (CYP11A1); aldo-keto reductase family 1, member C1 (AKRIC1); aldo-keto reductase family 1, member C2 (AKR1C2); aldo-keto reductase family 1, member C3 (AKR1C3); aldo-keto reductase family 1, member C4 (AKR1C4); 3β-hydroxysteroid dehydrogenase (3-β-HSD (e.g., hydroxy-delta-5-steroid dehydrogenase, 3 beta- and steroid delta-isomerase 2 (HSD3B2)); and Steroidogenic acute regulatory protein (StAR); and/or
wherein the neuroactive steroid-based agent is selected from the group consisting of steroid 5 alpha-reductase 1 (SRD5A1); steroid 5 alpha-reductase 2 (SRD5A2); translocator protein (TSPO); cytochrome P450 family 11 subfamily A member 1 (CYP11A1); aldo-keto reductase 1C superfamily; 3β-hydroxysteroid dehydrogenase (3-β-HSD); Steroidogenic acute regulatory protein (StAR); positive allosteric modulator of GABAA receptors; brexanolone (ZULRESSO®); zuranolone (ZURZUVAE™)); progesterone analogue; allopregnanolone prodrug; brexanolone prodrug; and delta-subunit-selective GABAA receptor modulator.


SEQUENCE LISTING (27 sequences)

SEQ ID NO: 1 - DNA, 21 bp, synthetic construct
  source 1..21: mol_type = other DNA
cagaccctgtctgctgttatc

SEQ ID NO: 2 - DNA, 21 bp, synthetic construct
  source 1..21: mol_type = other DNA
gataacagcagacagggtctg

SEQ ID NO: 3 - DNA, 20 bp, synthetic construct
  source 1..20: mol_type = other DNA
gagatattcagctgagaccc

SEQ ID NO: 4 - DNA, 20 bp, synthetic construct
  source 1..20: mol_type = other DNA
ttagtatgtgggcagcttgg

SEQ ID NO: 5 - DNA, 22 bp, synthetic construct
  source 1..22: mol_type = other DNA
ccctttgagggtcagatgtatg

SEQ ID NO: 6 - DNA, 22 bp, synthetic construct
  source 1..22: mol_type = other DNA
catacatctgaccctcaaaggg

SEQ ID NO: 7 - DNA, 20 bp, synthetic construct
  source 1..20: mol_type = other DNA
atttgtgtggcagagagagg

SEQ ID NO: 8 - DNA, 20 bp, synthetic construct
  source 1..20: mol_type = other DNA
ttgattgactgcctggatgg

SEQ ID NO: 9 - DNA, 21 bp, synthetic construct
  source 1..21: mol_type = other DNA
tcctacctggtctggaaagag

SEQ ID NO: 10 - DNA, 21 bp, synthetic construct
  source 1..21: mol_type = other DNA
ctctttccagaccaggtagga

SEQ ID NO: 11 - DNA, 21 bp, synthetic construct
  source 1..21: mol_type = other DNA
ccagaagtatggcccgattta

SEQ ID NO: 12 - DNA, 21 bp, synthetic construct
  source 1..21: mol_type = other DNA
taaatcgggccatacttctgg

SEQ ID NO: 13 - DNA, 21 bp, synthetic construct
  source 1..21: mol_type = other DNA
gggcaggtgacaggtatttat

SEQ ID NO: 14 - DNA, 21 bp, synthetic construct
  source 1..21: mol_type = other DNA
ataaatacctgtcacctgccc

SEQ ID NO: 15 - DNA, 22 bp, synthetic construct
  source 1..22: mol_type = other DNA
cgggttccaccatattgattct

SEQ ID NO: 16 - DNA, 22 bp, synthetic construct
  source 1..22: mol_type = other DNA
agaatcaatatggtggaacccg

SEQ ID NO: 17 - DNA, 22 bp, synthetic construct
  source 1..22: mol_type = other DNA
ccagaggttccgagaagtaaag

SEQ ID NO: 18 - DNA, 22 bp, synthetic construct
  source 1..22: mol_type = other DNA
ctttacttctcggaacctctgg

SEQ ID NO: 19 - DNA, 22 bp, synthetic construct
  source 1..22: mol_type = other DNA
ggaggtcatggagaagtgtaag

SEQ ID NO: 20 - DNA, 22 bp, synthetic construct
  source 1..22: mol_type = other DNA
cttacacttctccatgacctcc

SEQ ID NO: 21 - DNA, 23 bp, synthetic construct
  source 1..23: mol_type = other DNA
actcagcccaatttactcctatc

SEQ ID NO: 22 - DNA, 23 bp, synthetic construct
  source 1..23: mol_type = other DNA
gataggagtaaattgggctgagt

SEQ ID NO: 23 - DNA, 24 bp, synthetic construct
  source 1..24: mol_type = other DNA
cggagtcaacggatttggtcgtat

SEQ ID NO: 24 - DNA, 24 bp, synthetic construct
  source 1..24: mol_type = other DNA
atacgaccaaatccgttgactccg

SEQ ID NO: 25 - DNA, 22 bp, synthetic construct
  source 1..22: mol_type = other DNA
aggaggagatgggtacaactat

SEQ ID NO: 26 - DNA, 22 bp, synthetic construct
  source 1..22: mol_type = other DNA
agcacttaagctctggctattt

SEQ ID NO: 27 - DNA, 24 bp, synthetic construct
  source 1..24: mol_type = other DNA
agccttctccatggtggtgaagac